(19) United States
(12) Patent Application Publication
(10) Pub. No.: US 2008/0214654 A1
(43) Pub. Date: Sep. 4, 2008

(54) Substituted Benzyloxy-Phenylmethylamide Derivatives

(75) Inventors: Thomas Lampe, Dusseldorf (DE); Cristina Alonso-Alija, Haan (DE); Beatrix Stelte-Ludwig, Wulfrath (DE); Peter Sandner, Wuppertal (DE); Marcus Bauser, Wuppertal (DE); Hartmut Beck, Koln (DE); Klemens Lustig, Wuppertal (DE); Ulrich Rosentreter, Wunstorf (DE); Elke Stahl, Bergisch Gladbacht (DE); Hiroko Takagi, Dusseldorf (DE)

Correspondence Address: Barbara A. Shimei;Director, Patents & Licensing, Bayer HealthCare LLC - Pharmaceuticals, 555 White Plains Road, Third Floor, Tarrytown, NY, 10591, US

(73) Assignee: Bayer HealthCare AG, Leverkusen (DE)

(21) Appl. No.: 11/665,316

(22) PCT Filed: Oct. 12, 2005
(86) PCT No.: PCT/EP05/10951

§ 371 Date: Sep. 13, 2007

(30) Foreign Application Priority Data
Oct. 13, 2004 (EP) 04024362.8
Oct. 13, 2004 (EP) 04024363.6
Jul. 15, 2005 (GB) 0514579.2

(51) Int. Cl. A61K 31/381; C07C 271/06; A61K 31/27; A61K 31/164; A61P 13/02; C07C 237/00; C07D 333/02

(52) U.S. Cl. 514/448; 560/115; 514/478; 549/72; 564/184; 514/617

(57) ABSTRACT

The present invention relates to novel substituted benzyloxy-phenylmethylamide derivatives, processes for their preparation, and their use in medicaments, especially for the prophylaxis and treatment of diseases associated with Cold Menthol Receptor 1 (CMR-1) activity, in particular for the treatment of urological diseases or disorders, such as detrusor overactivity (overactive bladder), urinary incontinence, neurogenic detrusor oeractivity (detrusor hyperflexia), idiopathic detrusor overactivity (detrusor instability), benign prostatic hyperplasia, and lower urinary tract symptoms; chronic pain, neuropathic pain, postoperative pain, rheumatoid arthritic pain, neuralgia, neuropathies, algesia, nerve injury, ischaemia, neurodegeneration, stroke, and inflammatory disorders such as asthma and chronic obstructive pulmonary (or airways) disease (COPD).

The present invention relates to novel substituted benzyloxy-phenylmethylamide derivatives, processes for their preparation, and their use in medicaments, especially for the prophylaxis and treatment of diseases associated with Cold Menthol Receptor 1 (CMR-1) activity, in particular for the treatment of urological diseases or disorders, such as detrusor overactivity (overactive bladder), urinary incontinence, neurogenic detrusor overactivity (detrusor hyperflexia), idiopathic detrusor overactivity (detrusor instability), benign prostatic hyperplasia, and lower urinary tract symptoms; chronic pain, neuropathic pain, postoperative pain, rheumatoid arthritic pain, neuralgia, neuropathies, algesia, nerve injury, ischaemia, neurodegeneration, stroke, and inflammatory disorders such as asthma and chronic obstructive pulmonary (or airways) disease (COPD).

There is abundant direct or indirect evidence that shows the relation between Transient Receptor Potential (TRP) channel activity and diseases such as pain, ischaemia, and inflammatory disorders. Further, it has been demonstrated that TRP channels transduce reflex signals that are involved in the overactive bladder of patients who have damaged or abnormal spinal reflex pathways [De Groat WC: A neurologic basis for the overactive bladder. Urology 50 (6A Suppl): 36-52, 1997]. CMR-1, a nonselective cation channel is such a member of the TRP channel family (TRPM8). Recently, in 2002 the receptor was cloned and it was found to be sensitive to cold temperature and menthol and therefore named as cold menthol receptor-1 (CMR-1) (McKemy et al, 2002; Peier et al., 2002). This receptor which is activated by 8-28° C. temperature is expressed on the bladder urothelium and DRG (Dorsal Root Ganglia) and C-fibers. The intravesical ice water or menthol also induce C-fiber mediated spinal micturition reflex in patients with urgency and urinary incontinence (UI). Clinically CMR-1 is supposed to mediate the bladder cooling reflex seen after ice water test in overactive patients.

Therefore antagonism of the CMR-1 receptor leads to the blockage of neurotransmitter release, resulting in prophylaxis and treatment of the conditions and diseases associated with CMR-1 activity.

Antagonists of the CMR-1 receptor can be used for prophylaxis and treatment of the conditions and diseases including chronic pain, neuropathic pain, postoperative pain, rheumatoid arthritic pain, neuralgia, neuropathies, algesia, nerve injury, ischaemia, neurodegeneration, stroke, inflammatory disorders, urinary incontinence (U) such as urge urinary incontinence (UUI), and/or overactive bladder, Lower urinary tract symptoms secondary to or independent of benign prostatic hyperplasia.

UI is the involuntary loss of urine. UUI is one of the most common types of UI together with stress urinary incontinence (SUI) which is usually caused by a defect in the urethral closure mechanism. UUI is often associated with neurological disorders or diseases causing neuronal damages such as dementia, Parkinson's disease, multiple sclerosis, stroke and diabetes, although it also occurs in individuals with no such disorders. One of the usual causes of UUI is overactive bladder (OAB) which is a medical condition referring to the symptoms of frequency and urgency derived from abnormal contractions and instability of the detrusor muscle.

There are several medications for urinary incontinence on the market today mainly to help treating UUI. Therapy for OAB is focused on drugs that affect peripheral neural control mechanisms or those that act directly on bladder detrusor smooth muscle contraction, with a major emphasis on development of anticholinergic agents. These agents can inhibit the parasympathetic nerves which control bladder voiding or can exert a direct spasmolytic effect on the detrusor muscle of the bladder. This results in a decrease in intravesicular pressure, an increase in capacity and a reduction in the frequency of bladder contraction. Orally active anticholinergic drugs which are commonly prescribed have serious drawbacks such as unacceptable side effects such as dry mouth, abnormal visions, constipation, and central nervous system disturbances. These side effects lead to poor compliance. Dry mouth symptoms alone are responsible for a 70% non-compliance rate with oxybutynin. The inadequacies of present therapies highlight the need for novel, efficacious, safe, orally available drugs that have fewer side effects.

In WO 03/037865 and Y. Lu, et al., Bioorg. Med. Chem. Lett. 2004, 14, 3957-3962 related benzyloxy-phenylmethylamide derivatives for the treatment of cancer are described.

The present invention relates to compounds of the general formula (I)

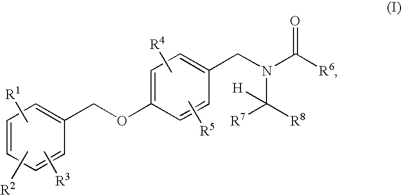

wherein
R¹ represents hydrogen or halogen,
R² represents hydrogen or halogen,
R³ represents hydrogen or halogen,
R⁴ represents chlorine, trifluoromethoxy or C₁-C₆-alkoxy,
R⁵ represents hydrogen, halogen, trifluoromethoxy, C₁-C₆-alkyl or C₁-C₆-alkoxy,
R⁶ represents C₃-C₈-alkyl, C₂-C₆-alkenyl, C₂-C₆-alkinyl, C₃-C₇-cycloalkyl, tetrahydronaphthyl, phenyl, 5- to 10-membered heteroaryl or a group of the formula —Y—R⁹,
   wherein cycloalkyl can be further substituted with one to three identical or different radicals selected from the group consisting of C₁-C₄-alkyl, and
   wherein phenyl and heteroaryl can be further substituted with one to three identical or different radicals selected from the group consisting of halogen, amino, hydroxy, trifluoromethyl, C₁-C₆-alkyl, C₁-C₆-alkoxy and C₁-C₆-alkylamino, and
   wherein
   Y represents C₁-C₄-alkandiyl,
   R⁹ represents C₃-C₇-cycloalkyl, phenyl or 5- to 10-membered heteroaryl,
      wherein cycloalkyl can be further substituted with one to three identical or different radicals selected from the group consisting of C₁-C₄-alkyl, and
      wherein phenyl and heteroaryl can be further substituted with one to three identical or different radicals selected from the group consisting of halogen, amino, hydroxy, trifluoromethyl, C₁-C₆-alkyl, C₁-C₆-alkoxy and C₁-C₆-alkylamino,
R⁷ represents C₁-C₆-alkyl, C₃-C₇-cycloalkyl or phenyl,
   wherein alkyl is further substituted with one radical selected from the group consisting of amino, mono-alkylamino, C₁-C₄-alkylcarbonylamino, C₁-C₄-alkoxycarbonylamino, phenyl or optionally C₁-C₄-alkyl substituted C₃-C₇-cycloalkyl, and
   wherein cycloalkyl and phenyl can be further substituted with one to three identical or different radicals selected from the group consisting of halogen, amino, hydroxy, trifluoromethyl, C₁-C₆-alkyl, C₁-C₆-alkoxy and C₁-C₆-alkylamino,
R⁸ represents hydrogen or C₁-C₄-alkyl,
   and their salts, hydrates and/or solvates.

Physiologically acceptable salts are preferred in the context of the present invention.

Physiologically acceptable salts according to the invention are non-toxic salts which in general are accessible by reaction of the compounds (I) with an inorganic or organic base or acid conventionally used for this purpose. Non-limiting examples of pharmaceutically acceptable salts of compounds (I) include the alkali metal salts, e.g. lithium, potassium and sodium salts, the alkaline earth metal salts such as magnesium and calcium salts, the quaternary ammonium salts such as, for example, triethyl ammonium salts, acetates, benzene sulphonates, benzoates, dicarbonates, disulphates, ditartrates, borates, bromides, carbonates, chlorides, citrates, dihydrochlorides, fumarates, gluconates, glutamates, hexyl resorcinates, hydrobromides, hydrochlorides, hydroxynaphthoates, iodides, isothionates, lactates, laurates, malates, maleates, mandelates, mesylates, methylbromides, methylnitrates, methylsulphates, nitrates, oleates, oxalates, palmitates, pantothenates, phosphates, diphosphates, polygalacturonates, salicylates, stearates, sulphates, succinates, tartrates, tosylates, valerates, and other salts used for medicinal purposes.

Hydrates of the compounds of the invention or their salts are stoichiometric compositions of the compounds with water, such as for example hemi-, mono-, or dihydrates.

Solvates of the compounds of the invention or their salts are stoichiometric compositions of the compounds with solvents.

The present invention includes both the individual enantiomers or diastereomers and the corresponding racemates or diastereomeric mixtures of the compounds according to the invention and their respective salts. In addition, all possible tautomeric forms of the compounds described above are included according to the present invention. The diastereomeric mixtures can be separated into the individual isomers by chromatographic processes. The racemates can be resolved into the respective enantiomers either by chromatographic processes on chiral phases or by resolution.

In the context of the present invention, the substituents, if not stated otherwise, in general have the following meaning:

Alkyl in general represents a straight-chain or branched saturated hydrocarbon radical having 1 to 6, preferably 1 to 4 carbon atoms. Non-limiting examples include methyl, ethyl, n-propyl, iso-propyl, n-butyl, isobutyl, sec-butyl, tert-butyl, pentyl, isopentyl, hexyl, isohexyl. The same applies to radicals such as alkoxy, alkylamino, alkylcarbonylamino, alkoxycarbonylamino and the like.

Alkandiyl in general represents a straight-chain or branched saturated alkandiyl radical having 1 to 4 carbon atoms. Non-limiting examples include methylen, ethan-1,2-diyl, ethan-1,1-diyl, propan-1,3-diyl, propan-1,2-diyl, propan-2,2-diyl, butan-1,4-diyl, butan-1,3-diyl and butan-2,4-diyl.

Alkenyl in general represents a straight-chain or branched alkenyl radical having 2 to 6, preferably 2 to 4 carbon atoms. Non-limiting examples include vinyl, allyl, n-prop-1-en-1-yl, n-but-2-en-1-yl, 2-methylprop-1-en-1-yl and 2-methylprop-2-en-1-yl.

Alkinyl in general represents a straight-chain or branched alkinyl radical having 2 to 6, preferably 2 to 4 carbon atoms. Non-limiting examples include ethinyl, propargyl (2-propinyl), 1-propinyl, but-1-inyl, but-2-inyl.

Alkoxy illustratively and preferably represents methoxy, ethoxy, n-propoxy, isopropoxy, tert-butoxy, n-pentoxy and n-hexoxy.

Alkylcarbonylamino in general represents a straight-chain or branched hydrocarbon radical having 1 to 6, preferably 1 to 4 carbon atoms which has a carbonylamino (—CO—NH—) function at the position of attachment and which is bonded to the carbonyl group. Non-limiting examples include formylamino, acetylamino, n-propionylamino, n-butyrylamino, isobutyrylamino, pivaloylamino, n-hexanoylamino.

Alkoxycarbonylamino illustratively and preferably represents methoxycarbonylamino, ethoxycarbonylamino, n-propoxycarbonylamino, isopropoxycarbonylamino, tert-butoxycarbonylamino, n-pentoxycarbonylamino and n-hexoxycarbonylamino.

Alkylamino represents an alkylamino radical having one or two (independently selected) alkyl substituents, illustratively and preferably representing methylamino, ethylamino, n-propylamino, isopropylamino, tert-butylamino, n-pentylamino, n-hexylamino, N,N-dimethylamino, N,N-diethylamino, N-ethyl-N-methylamino, N-methyl-N-n-propylamino, N-isopropyl-N-n-propylamino, N-tert-butyl-N-methylamino, N-ethyl-N-n-pentylamino and N-n-hexyl-N-methylamino.

Mono-alkylamino represents an alkylamino radical having one alkyl substituents, illustratively and preferably representing methylamino, ethylamino, n-propylamino, isopropylamino, tert-butylamino, n-pentylamino and n-hexylamino.

Cycloalkyl in general represents a cyclic saturated hydrocarbon radical having 3 to 8, preferably 3 to 6 carbon atoms. Non-limiting examples include cyclopropyl, cyclobutyl, cyclopentyl, cyclohexyl and cycloheptyl.

Heteroaryl per se and in heteroarylmethyl in general represents an aromatic mono- or bicyclic radical having 5 to 10 and preferably 5 or 6 ring atoms, and up to 5 and preferably up to 4 heteroatoms selected from the group consisting of S, O and N, illustratively and preferably representing thienyl, furyl, pyrrolyl, pyrazolyl, thiazolyl, oxazolyl, isothiazolyl, isoxazolyl, imidazolyl, oxadiazolyl, thiadiazolyl, pyridyl, pyrimidyl, pyridazinyl, indolyl, indazolyl, benzofuranyl, benzothienyl, benzothiazolyl, quinolinyl, isoquinolinyl.

Halogen represents fluorine, chlorine, bromine and iodine.

In another preferred embodiment, the present invention relates to compounds of general formula (I), wherein
R¹ represents hydrogen or halogen,
R² represents hydrogen or halogen,
R³ represents hydrogen,
R⁴ represents C₁-C₆-alkoxy,
R⁵ represents hydrogen,
R⁶ represents C₃-C₈-alkyl, C₂-C₆-alkenyl, C₃-C₇-cycloalkyl, tetrahydronaphthyl, phenyl, 5- to 6-membered heteroaryl or a group of the formula —Y—R⁹,
   wherein cycloalkyl can be further substituted with one to three identical or different radicals selected from the group consisting of C₁-C₄-alkyl, and
   wherein phenyl and heteroaryl can be further substituted with one to three identical or different radicals selected from the group consisting of halogen, trifluoromethyl, C₁-C₆-alkyl and C₁-C₆-alkoxy, and
   wherein
   Y represents C₁-C₄-alkandiyl,
   R⁹ represents C₃-C₇-cycloalkyl, phenyl or 5- to 6-membered heteroaryl,
      wherein cycloalkyl can be further substituted with one to three identical or different radicals selected from the group consisting of C₁-C₄-alkyl, and
      wherein phenyl and heteroaryl can be further substituted with one to three identical or different radicals selected from the group consisting of halogen, trifluoromethyl, C₁-C₆-alkyl and C₁-C₆-alkoxy,
R⁷ represents C₁-C₄-alkyl, C₃-C₆-cycloalkyl or phenyl,
   wherein alkyl is further substituted with one radical selected from the group consisting of amino, mono-alkylamino, C₁-C₄-alkoxycarbonylamino, phenyl or optionally C₁-C₄-alkyl substituted C₃-C₆-cycloalkyl, and
   wherein cycloalkyl and phenyl can be further substituted with one to three identical or different radicals selected from the group consisting of amino, C₁-C₆-alkyl, C₁-C₆-alkoxy and C₁-C₆-alkylamino,
R⁵ represents hydrogen,
   and their salts, hydrates and/or solvates.

In another particularly preferred embodiment, the present invention relates to compounds of general formula (I), wherein
R¹ represents hydrogen, fluorine or chlorine,
R² represents hydrogen or fluorine,
R³ represents hydrogen,
R⁴ represents methoxy,
R⁵ represents hydrogen,
R⁶ represents C₃-C₆-alkyl, C₃-C₆-cycloalkyl, tetrahydronaphthyl, phenyl, thienyl, furyl, pyrazolyl or a group of the formula —Y—R⁹,
   wherein cycloalkyl can be further substituted with one or two methyl groups, and
   wherein phenyl, thienyl, furyl and pyrazolyl can be further substituted with one to three identical or different radicals selected from the group consisting of fluorine, chlorine, trifluoromethyl, methyl and methoxy, and
   wherein
   Y represents methylen,
   R⁹ represents C₃-C₆-cycloalkyl, thienyl, furyl or pyrazolyl,
      wherein cycloalkyl can be further substituted with one or two methyl groups, and
      wherein thienyl, furyl and pyrazolyl can be further substituted with one to three identical or different radicals selected from the group consisting of fluorine, chlorine, trifluoromethyl, methyl and methoxy,
R⁷ represents C₁-C₂-alkyl, cyclopropyl, cyclohexyl or phenyl,
   wherein alkyl is further substituted with one radical selected from the group consisting of amino or tert-butoxycarbonylamino,
R⁸ represents hydrogen,
   and their salts, hydrates and/or solvates.

In another preferred embodiment, the present invention relates to compounds of general formula (I), wherein R⁷ represents —CH₂NH₂ or —CH₂CH₂NH₂.

In another preferred embodiment, the present invention relates to compounds of general formula (I), wherein R¹, R² and R³ represent hydrogen.

In another preferred embodiment, the present invention relates to compounds of general formula (I), wherein R¹ represents halogen, R² represents hydrogen or halogen and R³ represents hydrogen or halogen.

In another preferred embodiment, the present invention relates to compounds of general formula (I), wherein R¹ represents halogen, R² represents hydrogen or halogen and R³ represents hydrogen.

In another preferred embodiment, the present invention relates to compounds of general formula (I), wherein R¹ represents fluorine or chlorine, R² represents hydrogen or fluorine and R³ represents hydrogen.

In another preferred embodiment, the present invention relates to compounds of general formula (I), wherein R⁴ represents trifluoromethoxy or C₁-C₆-alkoxy.

Very particular preference is given to combinations of two or more of the above-mentioned preference ranges.

The compounds of general formula (I) can be synthesized by condensing compounds of general formula (II)

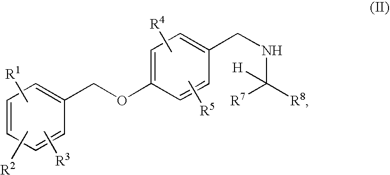

wherein R¹, R², R³, R⁴, R⁵, R⁷ and R⁸ have the meaning indicated above, with compounds of general formula (III)

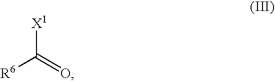

wherein R⁶ has the meaning indicated above, and
X¹ represents a leaving group, such as halogen, preferably chlorine or bromine, or hydroxy,
   in the presence of a base.

Amino groups in R⁷ of compounds of general formula (II) are protected with acid labile groups, preferred is a boc-group. After the synthesis of compounds of general formula (I) this acid labile group can be cleaved via standard procedures known by a person skilled in the art. Compounds of general formula (I) are obtained. Preferred are acidic cleavage conditions.

If a salt of a compound of general formula (1), for example a hydrochloride or trifluoroacetate, is isolated the free base can be obtained by reversed phase chromatography of the salt using a mixture of acetonitile and water as eluent in the presence of a base. Preferably a RP18 Phenomenex Luna C18(2) column is used in the presence of diethylamine as base. Or the free base of a compound of general formula (I) can be obtained by neutralizing with a base and extraction.

The process is in general carried out in a temperature range from −20° C. to boiling point of the solvent, preferably from 0° C. to +40° C.

The process is generally carried out at normal pressure. However, it is also possible to carry it out at elevated pressure or at reduced pressure (for example in a range from 0.5 to 5 bar).

Suitable solvents for the process are ethers such as dioxan or tetrahydrofuran, or halogeno-hydrocarbons such as dichloromethane, dichloroethane or trichloromethane, or other solvents such as dimethylformamide, ethyl acetate or acetonitrile. It is also possible to use mixtures of the above-mentioned solvents. Preferred for the process is tetrahydrofuran or dichloromethane. Suitable bases for the process are generally inorganic or organic bases. These preferably include alkali carbonates such as sodium or potassium carbonate or hydrogencarbonate, cyclic amines such as, for example, N-methylmorpholine, N-methylpiperidine, pyridine or 4-N,N-dimethylaminopyridine, or (C₁-C₄)-trialkylamines such as, for example, triethylamine or diisopropylethylamine. Preference is given to triethylamine.

If X¹ is hydroxy, a coupling agent is added to the reaction mixture such as a carbodiimide, for example N,N′-diethyl-, N,N,′-dipropyl-, N,N′-diisopropyl-, N,N′-dicyclohexylcarbodiimide, N-(3-di-methylaminoisopropyl)-N′-ethylcarbodiimide-hydrochloride (EDC), N-cyclohexylcarbodiimide-N′-propyloxymethyl-polystyrene (PS-carbodiimide) or O-(benzotriazol-1-yl)-N,N,N′,N′-tetramethyl-uronium-hexafluorophosphate (HBTU), 2-(2-oxo-1-(2H)-pyridyl)-1,1,3,3-tetramethyluroniumtetrafluoroborate (TPTU) or O-(7-azabenzotriazol-1-yl)-N,N,N′,N′-tetramethyl-uronium-hexafluorophosphate (HATU), or 1-hydroxybenztriazole (HOBt), or benzotriazol-1-yloxytris(dimethylamino)-phosphoniumhexafluorophosphate (BOP), or mixtures of these reagents.

The compounds of the general formula (M) are known per se, or they can be prepared by customary methods.

The compounds of general formula (II) can be synthesized by condensing compounds of general formula (IV)

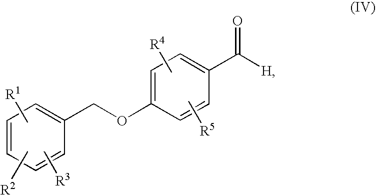

wherein R¹, R², R³, R⁴ and R⁵ have the meaning indicated above,
with compounds of general formula (V)

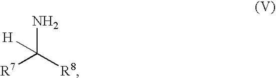

wherein R⁷ and R⁸ have the meaning indicated above,
under conditions of a reductive amination.

The process is in general carried out in a temperature range from −20° C. to boiling point of the solvent, preferably from 0° C. to +40° C.

The process is generally carried out at normal pressure. However, it is also possible to carry it out at elevated pressure or at reduced pressure (for example in a range from 0.5 to 5 bar).

Suitable solvents for the process are halogeno-hydrocarbons such as dichloromethane, dichloroethane or trichloromethane, or alcohols such as methanol, ethanol, n-propanol, iso-propanol, n-butanol or tert-butanol, or a mixture of alcohol and water. Preferred for the process is methanol or a mixture of methanol and water.

Suitable reducing agents for the process are sodium borohydride or triacetoxyborohydride.

The compounds of the general formula (V) are known per se, or they can be prepared by customary methods.

The compounds of general formula (IV) can be synthesized by condensing compounds of general formula (VI)

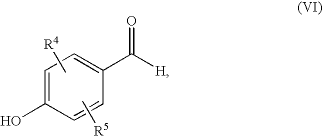

wherein R⁴ and R⁵ have the meaning indicated above,
with compounds of general formula (VII)

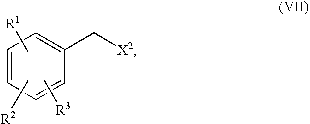

wherein R¹, R² and R³ have the meaning indicated above, and
X² represents a leaving group, such as halogen, preferably chlorine or bromine,
in the presence of a base.

Optionally an alkali iodide such as sodium or potassium iodide can be added to the reaction mixture.

The process is in general carried out in a temperature range from 0° C. to boiling point of the solvent, preferably from 20° C. to boiling point of the solvent.

The process is generally carried out at normal pressure. However, it is also possible to carry it out at elevated pressure or at reduced pressure (for example in a range from 0.5 to 5 bar).

Suitable solvents for the process are ethers such as dioxan or tetrahydrofuran, or halogeno-hydrocarbons such as dichloromethane, dichloroethane or trichloromethane, or other solvents such as dimethylformamide, dimethylsulfoxide, ethyl acetate or acetonitrile. It is also possible to use mixtures of the above-mentioned solvents. Preferred for the process is acetonitrile.

Suitable bases for the process are generally inorganic or organic bases. These preferably include alkali carbonates such as sodium or potassium carbonate or hydrogencarbonate, cyclic amines such as, for example, N-methylmorpholine, N-methylpiperidine, pyridine or 4-N,N-dimethylaminopyridine, or (C₁-C₄)-trialkylamines such as, for example, triethylamine or diisopropylethylamine. Preference is given to potassium carbonate.

The compounds of the general formulas (VI) and (VII) are known per se, or they can be prepared by customary methods.

The above-mentioned process can be illustrated by the following scheme:

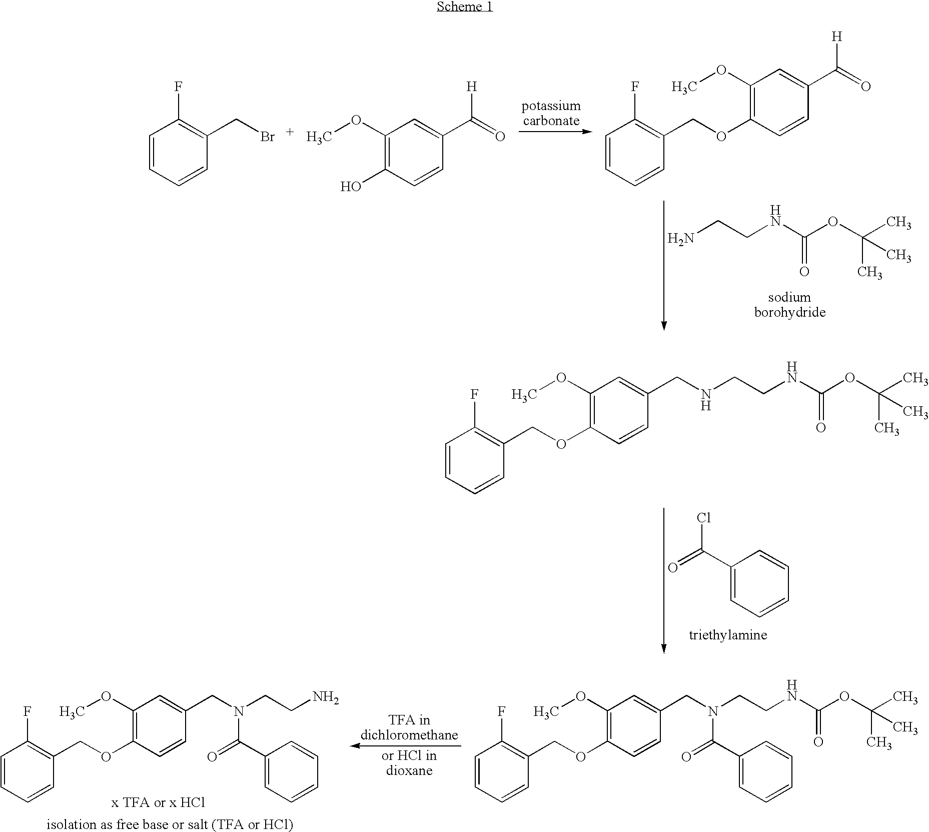

Alternatively above mentioned process can be conducted on solid support using polymer bound diamines. Initially the diamines are attached to the resin via an acid labile linkage. In the final step of the synthesis the products are released from the solid support. The following scheme illustrates the process on solid phase:

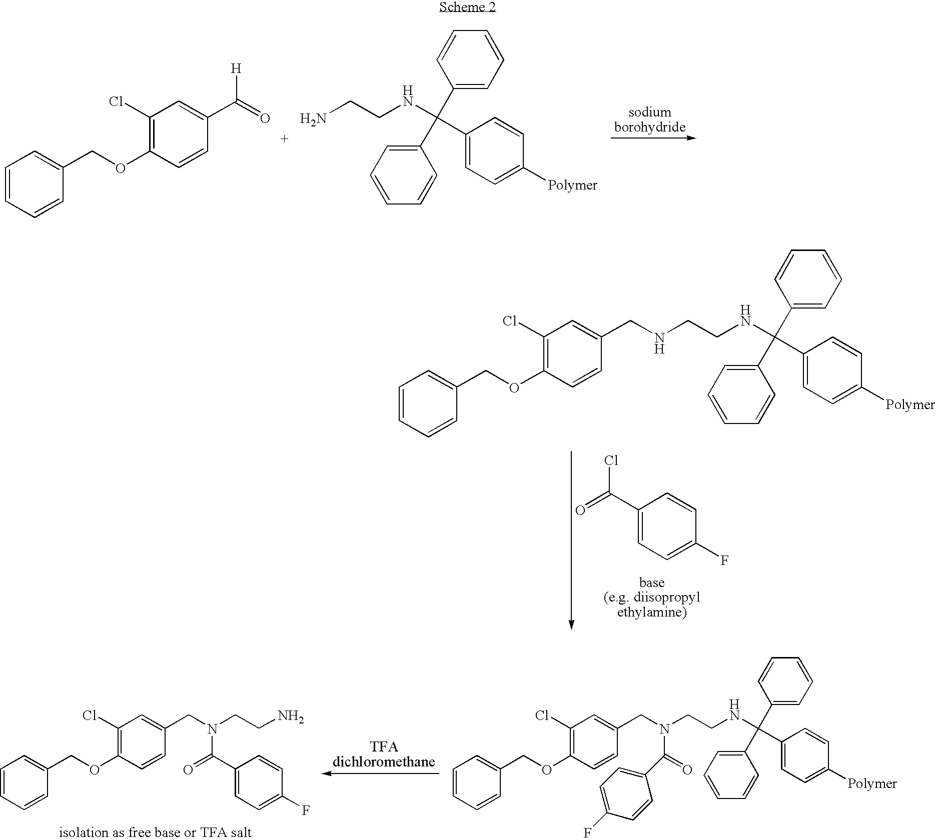

The compounds according to the invention exhibit an unforeseeable, useful pharmacological activity spectrum. They are therefore suitable for use as medicaments for the treatment and/or prophylaxis of disorders in humans and animals.

Surprisingly, the compounds of the present invention show excellent CMR-1 antagonistic activity. They are, therefore suitable especially for the prophylaxis and treatment of diseases associated with CMR-1 activity, in particular for the treatment of urological diseases or disorders, such as detrusor overactivity (overactive bladder), urinary incontinence, neurogenic detrusor oeractivity (detrusor hyperflexia), idiopathic detrusor overactivity (detrusor instability), benign prostatic hyperplasia, and lower urinary tract symptoms.

The compounds of the present invention are also effective for treating or preventing a disease selected from the group consisting of chronic pain, neuropathic pain, postoperative pain, rheumatoid arthritic pain, neuralgia, neuropathies, algesia, nerve injury, ischaemia, neuro-degeneration and/or stroke, as well as respiratory diseases and inflammatory diseases such as asthma, COPD and allergic rhinitis since the diseases also relate to CMR-1 activity.

The compounds of the present invention are also useful for the treatment and prophylaxis of neuropathic pain, which is a form of pain often associated with herpes zoster and post-herpetic neuralgia, painful diabetic neuropathy, neuropathic low back pain, posttraumatic and postoperative neuralgia, neuralgia due to nerve compression and other neuralgias, phantom pain, complex regional pain syndromes, infectious or parainfectious neuropathies like those associated with HIV infection, pain associated with central nervous system disorders like multiple sclerosis or Parkinson disease or spinal cord injury or traumatic brain injury, and post-stroke pain.

Furthermore, the compounds of the present invention are useful for the treatment of musculo-skeletal pain, forms of pain often associated with osteoarthritis or rheumatoid arthritis or other forms of arthritis, and back pain.

In addition, the compounds of the present invention are useful for the treatment of pain associated with cancer, including visceral or neuropathic pain associated with cancer or cancer treatment.

The compounds of the present invention are furthermore useful for the treatment of visceral pain, e.g. pain associated with obstruction of hollow viscus like gallstone colik, pain associated with irritable bowel syndrome, pelvic pain, vulvodynia, orchialgia or prostatodynia, pain associated with inflammatory lesions of joints, skin, muscles or nerves, and orofascial pain and headache, e.g. migraine or tension-type headache.

The present invention further provides medicaments containing at least one compound according to the invention, preferably together with one or more pharmacologically safe excipient or carrier substances, and also their use for the above-mentioned purposes.

The active component can act systemically and/or locally. For this purpose, it can be applied in a suitable manner, for example orally, parenterally, pulmonally, nasally, sublingually, lingually, buccally, rectally, transdermally, conjunctivally, otically or as an implant.

For these application routes, the active component can be administered in suitable application forms.

Useful oral application forms include application forms which release the active component rapidly and/or in modified form, such as for example tablets (non-coated and coated tablets, for example with an enteric coating), capsules, sugar-coated tablets, granules, pellets, powders, emulsions, suspensions, solutions and aerosols.

Parenteral application can be carried out with avoidance of an absorption step (intravenously, intraarterially, intracardially, intraspinally or intralumbarly) or with inclusion of an absorption (intramuscularly, subcutaneously, intracutaneously, percutaneously or intraperitoneally). Useful parenteral application forms include injection and infusion preparations in the form of solutions, suspensions, emulsions, lyophilisates and sterile powders.

Forms suitable for other application routes include for example inhalatory pharmaceutical forms (including powder inhalers, nebulizers), nasal drops/solutions, sprays; tablets or capsules to be administered lingually, sublingually or buccally, suppositories, ear and eye preparations, vaginal capsules, aqueous suspensions (lotions, shake mixtures), lipophilic suspensions, ointments, creams, milk, pastes, dusting powders or implants.

The active components can be converted into the recited application forms in a manner known per se. This is carried out using inert non-toxic, pharmaceutically suitable excipients. These include inter alia carriers (for example microcrystalline cellulose), solvents (for example liquid polyethylene glycols), emulsifiers (for example sodium dodecyl sulphate), dispersing agents (for example polyvinylpyrrolidone), synthetic and natural biopolymers (for example albumin), stabilizers (for example antioxidants such as ascorbic acid), colorants (for example inorganic pigments such as iron oxides) or taste and/or odor corrigents.

For human use, in the case of oral administration, it is recommendable to administer doses of from 0.001 to 50 mg/kg, preferably of 0.01 mg/kg to 20 mg/kg. In the case of parenteral administration, such as, for example, intravenously or via mucous membranes nasally, buccally or inhalationally, it is recommendable to use doses of 0.001 mg/kg to 0.5 mg/kg.

In spite of this, it can be necessary in certain circumstances to depart from the amounts mentioned, namely as a function of body weight, application route, individual behaviour towards the active component, manner of preparation and time or interval at which application takes place. It can for instance be sufficient in some cases to use less than the aforementioned minimum amount, while in other cases the upper limit mentioned will have to be exceeded. In the case of the application of larger amounts, it can be advisable to divide them into a plurality of individual doses spread through the day.

The percentages in the tests and examples which follows are, unless otherwise stated, by weight; parts are by weight. Solvent ratios, dilution ratios and concentrations reported for liquid/liquid solutions are each based on the volume.

### A. EXAMPLES

### Abbreviations

aq. aqueous
boc tert-butoxycarbonyl
c concentration
CDCl₃ deutero chloroform
conc. concentrated
DCI direct chemical ionisation (for MS)
DMAP 4-N,N-dimethylaminopyridine
DMF IVN-dimethylformamide
DMSO dimethylsulfoxide
El electron impact ionisation (for MS)
ESI electro-spray ionisation (for MS)
h hour(s)
HOBT hydroxybenzotriazole
HPLC high pressure liquid chromatography
LC-MS liquid chromatography coupled with mass spectroscopy
min minute(s)
Mp. melting point
MS mass spectroscopy
NMR nuclear magnetic resonance spectroscopy
of th. of theoretical (yield)
RP reverse phase (for HPLC)
Rₜ retention time (for HPLC)
sat. saturated
TFA trifluoroacetic acid
THF tetrahydrofuran

### LC-MS/HPLC Methods:

method 1 (HPLC): Instrument: HP 1100 with DAD detection; column: Kromasil 100 RP-18, 60 mm×2.1 mm, 3.5 μm; eluent A: 5 ml HClO₄/l water, eluent B: acetonitrile; gradient: 0 min 2% B→0.5 min 2% B→4.5 min 90% B→6.5 min 90% B→6.7 min 2% B→7.5 min 2% B; flow: 0.75 ml/min; oven: 30° C.; UV detection: 210 nm.

method 2 (LC-MS): Instrument: Micromass Quattro LCZ with HPLC Agilent Series 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min→2.5 min/3.0 min/4.5 min 2 ml/min; oven: 50° C.; UV detection: 208-400 nm n.

method 3 (LC-MS): Instrument MS: Micromass ZQ; Instrument HPLC: HP 1100 Series; UV DAD; column: Phenomenex Synergi 2μ Hydro-RP Mercury 20 mm×4 mm; eluent A: 1 l water+0.5 ml 50% formic acid, eluent B: 1 acetonitrile+0.5 ml 50% formic acid; gradient: 0.0 min 90% A→2.5 min 30% A→3.0 min 5% A→4.5 min 5% A; flow: 0.0 min 1 ml/min ˜2.5 min/3.0 min/4.5 min 2 ml/min; oven: 50° C.; UV detection: 210 nm.

method 4 (LC-MS): Instrument MS: Micromass ZQ; Instrument HPLC: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min→2.5 min/3.0 min/4.5 min 2 ml/min; oven: 50° C.; UV detection: 210 nm.

method 5 (HPLC): Instrument: HP 1100 with DAD detection; column: Kromasil 100 RP-18, 60 mm×2.1 mm, 3.5 g/m; eluent A: 5 ml HClO₄/l water, eluent B: acetonitrile; gradient: 0 min 2% B→0.5 min 2% B→4.5 min 90% B→9.0 min 90% B→9.2 min 2% B→10.0 min 2% B; flow: 0.75 ml/min; oven: 30° C.; UV detection: 210 nm.

method 6 (HPLC): Instrument MS: Micromass TOF (LCT); Instrument HPLC: Waters 2690; Column: YMC-ODS-AQ, 50 mm×4.6 mm, 3.0 μm; eluent A: water+0.1% formic acid, eluent B: acetonitrile+0.1% formic acid; gradient: 0.0 min 100% A→0.2 min 95% A→1.8 min 25% A→1.9 min 10% A→2.0 min 5% A→3.2 min 5% A; oven: 40° C.; flow: 3.0 ml/min; UV detection: 210 nm.

method 1-1 (LC-MS: Instrument MS: Micromass ZQ; Instrument HPLC: Waters Alliance 2795; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min→2.5 min/3.0 min/4.5 min 2 ml/min; oven: 50° C.; UV detection: 210 nm.

method 2-1 (HPLC): Instrument: HP 1100 with DAD detection; column: Kromasil 100 RP-18, 60 mm×2.1 mm, 3.5 μm; eluent A: 5 ml HClO₄/l water, eluent B: acetonitrile; gradient: 0 min 2% B→0.5 min 2% B→4.5 min 90% B→9.0 min 90% B→9.2 min 2% B→10.0 min 2% B; flow: 0.75 ml/min; oven: 30° C.; UV detection: 210 nm.

method 3-1 (LC-MS): Instrument MS: Micromass ZQ; Instrument HPLC: HP 1100 Series; UV DAD; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min→2.5 min/3.0 min/4.5 min 2 ml/min; oven: 50° C.; UV detection: 210 nm.

method 4-1 (LC-MS): Instrument: Micromass Quattro LCZ with HPLC Agilent Serie 1100; column: Phenomenex Synergi 2μ Hydro-RP Mercury 20 mm×4 mm; eluent A: 1 l water+0.5 ml 50% formic acid, eluent B: 1 l acetonitrile+0.5 ml 50% formic acid; gradient: 0.0 min 90% A→2.5 min 30% A→3.0 min 5% A→4.5 min 5% A; flow: 0.0 min 1 ml/min, 2.5 min/3.0 min/4.5 min 2 ml/min; oven: 50° C.; UV detection: 208-400 nm.

### Starting Materials and Intermediates:

### Example 1A

### 4-[(2-fluorobenzyl)oxy]-3-methoxybenzaldehyde

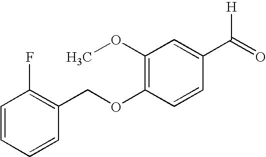

10.00 g (65.7 mmol) 4-hydroxy-3-methoxybenzaldehyde and 13.67 g (72.3 mmol) 2-fluorobenzyl bromide are dissolved in 100 ml acetonitrile. 45.4 g (328.6 mmol) potassium carbonate and 10.91 g (65.7 mmol) potassium iodide are added and the mixture is heated to reflux during 3 h. After cooling to room temperature, water is added and the solution is extracted twice with ethyl acetate. The combined organic materials are washed with water, dried over magnesium sulfate and the solvent is evaporated under vacuum. Petroleum ether is added, the solid is triturated, filtered and dried to yield 17.0 g (99% of th.) of the title compound.

HPLC (method 1): Rₜ=4.56 min

MS (DCI): m/z=261 (M+H)+

¹H-NMR (200 MHz, DMSO-d₆): δ=9.85 (s, 1H), 7.60-7.20 (m, 7H), 5.20 (s, 2H), 3.80 (s, 3H)

Using an analogous procedure the following compounds are prepared:

| Example | Starting materials | Structure | Characterisation |
|---|---|---|---|
| 2A | 4-hydroxy-3-methoxybenz-aldehyde and4-fluorobenzyl-bromide | 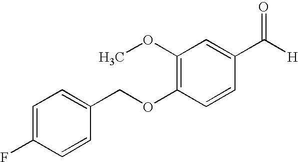 | ¹H-NMR (300 MHz,DMSO-d₆): δ = 9.85 (s, 1 H),7.60-7.50 (m, 3 H), 7.40 (d,1 H), 7.30-7.15 (m, 3 H), 5.20(s, 2 H), 3.80 (s, 3 H) |
| 3A | 4-hydroxy-3-methoxybenz-aldehyde and2,4-difluorobenzyl-bromide | 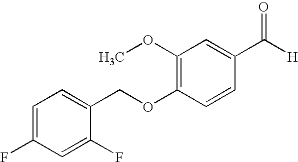 | ¹H-NMR (300 MHz,DMSO-d₆): δ 9.85 (s, 1 H),7.60-7.50 (m, 2 H), 7.40-7.10(m, 4 H), 5.20 (s, 2 H), 3.80(s, 3 H) |
| 4A | 4-hydroxy-3-methoxybenz-aldehyde and4-chlorobenzyl-bromide | 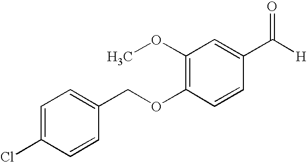 | ¹H-NMR (400 MHz,DMSO-d₆): δ = 9.85 (s, 1 H),7.55-7.40 (m, 6 H), 7.20 (d,1 H), 5.20 (s, 2 H), 3.80 (s,3 H) |

### Example 5A

### tert-butyl [2-({4-[(2-fluorobenzyl)oxy]-3-methoxybenzyl}amino)ethyl]carbamate

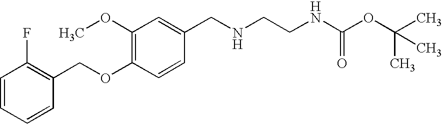

2.00 g (7.68 mmol) 4-[(2-fluorobenzyl)oxy]-3-methoxybenzaldehyde and 1.35 g (8.45 mmol) tert-butyl (2-aminoethyl)carbamate are dissolved in 40 ml methanol and stirred for 1 h at room temperature. The solution is cooled to 0° C. and 1.45 g (38.4 mmol) sodium borohydride are carefully added. Water is added until a clear solution is formed and the mixture is stirred during 2 h at room temperature. The mixture is concentrated under vacuum, the residue is diluted with dichloromethane and the organic phase is washed with brine, dried over magnesium sulfate and concentrated under vacuum to yield 2.40 g (68% of th.) of the title compound of sufficient purity to be used in the next step.

HPLC (method 1): Rₜ=4.47 min

MS (ESIpos): m/z=405 (M+H)⁺

¹H-NMR (300 MHz, DMSO-d₆): δ=7.55-7.20 (m, 4H), 6.90 (m, 2H), 6.80-6.70 (m, 2H), 5.05 (s, 2H), 3.75 (s, 3H), 3.60 (s, 2H), 3.00 (m, 2H), 2.50 (m, 2H), 1.40 (s, 9H)

### Example 6A

### tert-butyl [3-({4-[(4-fluorobenzyl)oxy]-3-methoxybenzyl}amino)propyl]carbamate

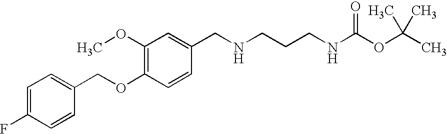

400.0 mg (1.54 mmol) 4-[(4-fluorobenzyl)oxy]-3-methoxybenzaldehyde and 294.6 mg (1.69 mmol) tert-butyl (2-aminopropyl)carbamate are dissolved in 70 ml methanol and stirred for 2 h at room temperature. The solution is cooled to 0° C. and 290.7 mg (7.68 mmol) sodium borohydride are carefully added. Water is added until a clear solution is formed and the mixture is stirred at room temperature overnight. The mixture is concentrated under vacuum, the residue is diluted with dichloromethane and the organic phase is washed with brine, dried over magnesium sulfate and concentrated under vacuum. The crude is purified by preparative HPLC (acetonitrile/water) to yield 287.0 g (45% of th.) of the title compound.

HPLC (method 1): Rₜ=4.55 min

MS (ESIpos): m/z=419 (M+H)⁺

¹H-NMR (400 MHz, DMSO-d₆): δ=7.50 (m, 2H), 7.20 (t, 2H), 6.90 (m, 2H), 6.80 (m, 2H), 5.00 (s, 2H), 3.75 (s, 3H), 3.60 (s, 2H), 2.95 (m, 2H), 2.45 (m, 2H), 2.50 (m, 2H), 1.40 (s, 9H)

Using an analogous procedure the following compounds are prepared:

| Example | Starting materials | Structure | Characterisation |
|---|---|---|---|
| 7A | 4-[(4-fluoro-benzyl)oxy]-3-methoxybenz-aldehyde andtert-butyl (2-amino-ethyl)carbamate | 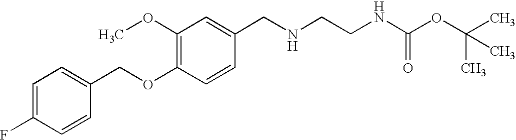 | ¹H-NMR (300 MHz,DMSO-d₆): δ = 7.50(m, 2 H), 7.20 (m, 2 H),6.90 (m, 2 H), 6.70 (m,2 H), 5.00 (s, 2 H), 3.75(s, 3 H), 3.60 (s, 2 H),3.00 (m, 2 H), 2.50 (m,2 H), 1.40 (s, 9 H) |
| 8A | 4-[(2,4-difluoro-benzyl)oxy]-3-methoxybenz-aldehyde andtert-butyl (2-amino-ethyl)carbamate | 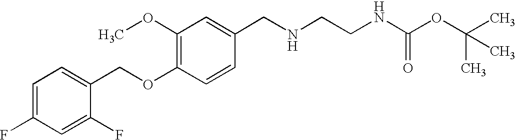 | ¹H-NMR (300 MHz,DMSO-d₆): δ =7.60(m, 1 H), 7.30 (m, 1 H),7.10 (m, 1 H), 6.95 (m,2 H), 6.80-6.70 (m, 2 H),5.00 (s, 2 H), 3.75 (s,3 H), 3.60 (s, 2 H), 3.00(m, 2 H), 2.50 (m, 2 H),1.40 (s, 9 H) |
| 9A | 4-[(4-chloro-benzyl)oxy]-3-methoxybenz-aldehyde andtert-butyl (2-amino-ethyl)carbamate | 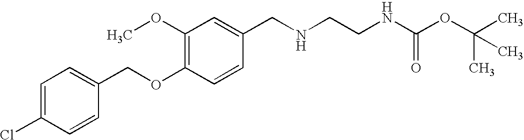 | ¹H-NMR (300 MHz,DMSO-d₆): δ =7.50 (s,4 H), 6.90 (m, 2 H), 6.80(m, 2 H), 5.00 (s, 2 H),3.75 (s, 3 H), 3.60 (s,2 H), 3.00 (m, 2 H), 2.90(m, 1 H), 2.50 (m, 2 H),1.40 (s, 9 H) |

### Example 10A

### N-benzyl-1-{4-[(4-fluorobenzyl)oxy]-3-methoxyphenyl}methanamine

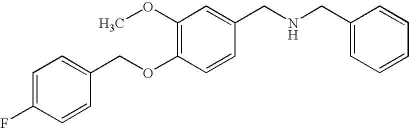

200.0 mg (0.768 mmol) 4-[(4-fluorobenzyl)oxy]-3-methoxybenzaldehyde and 90.6 mg (0.845 mmol) benzylamine are dissolved in 35 ml methanol and stirred for 2 h at room temperature. The solution is cooled to 0° C. and 145.4 mg (3.82 mmol) sodium borohydride are carefully added. Water is added until a clear solution is formed and the mixture is stirred at room temperature over 24 hours. The mixture is concentrated under vacuum, the residue is diluted with dichloromethane and the organic phase is washed with brine, dried over magnesium sulfate and concentrated under vacuum. The crude is purified by preparative HPLC (acetonitrile/water) to yield 66.0 mg (25% of th.) of the title compound.

HPLC (method 1): Rₜ=4.50 min

MS (ESIpos): m/z=352 (M+H)⁺

¹H-NMR (400 MHz, DMSO-d₆): δ=7.50 (m, 2H), 7.30 (m, 4H), 7.20 (m, 3H), 7.00 (m, 2H), 6.80 (d, 1H), 5.00 (s, 2H), 3.75 (s, 3H), 3.65 (s, 2H), 3.55 (s, 2H)

Using an analogous procedure the following compounds are prepared:

| Example | Starting materials | Structure | Characterisation |
|---|---|---|---|
| 11A | 4-[(4-fluorobenzyl)-oxy]-3-methoxy-benzaldehyde and1-cyclopropyl-methanamine | 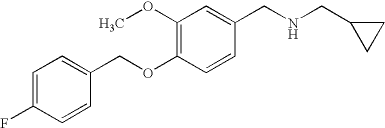 | LC/MS (method 2):Rₜ = 1.67 min,MS (ESIpos):m/z = 316 (M + H)⁺ |
| 12A | 4-[(4-fluorobenzyl)-oxy]-3-methoxy-benzaldehyde and1-cyclohexyl-methanamine | 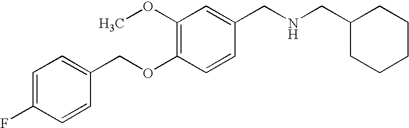 | ¹H-NMR (300 MHz,DMSO-d₆): δ = 7.50-6.70 (m, 7 H), 5.00 (s,2 H), 3.75 (s, 3 H), 3.60(s, 2 H), 2.25 (d, 2 H),1.80-0.80 (m, ^{11 H)} |

### Example 1-1A

### tert-butyl (2-{[4-(benzyloxy)-3-methoxybenzyl]amino}ethyl)carbamate

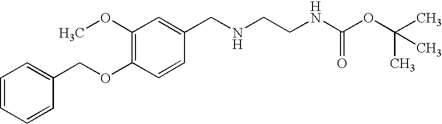

1.45 g (5.98 mmol) 4-benzyloxy-3-methoxybenzaldehyde and 1.01 g (6.58 mmol) tert-butyl (2-aminoethyl)carbamate are dissolved in 20 ml dichloromethane and stirred for 1 h at room temperature. The solution is cooled to 0° C. and 1.96 g (8.98 mmol) sodium triacetoxyborohydride are carefully added and the mixture is warmed to room temperature and stirred overnight. The reaction is quenched with water and the organic materials are washed with 1N sodium hydroxide solution and brine, dried over magnesium sulfate and concentrated under vacuum to yield 1.60 g (69% of th.) of the title compound of sufficient purity to be used in the next step.

LC-MS (method 1-1): Rₜ=1.44 min, m/z=387 (M+H)⁺

¹H-NMR (300 MHz, DMSO-d₆): δ=7.50-7.25 (m, 5H), 6.90 (m, 2H), 6.80-6.70 (m, 2H), 5.05 (s, 2H), 3.75 (s, 3H), 3.60 (s, 2H), 3.00 (m, 2H), 2.50 (m, 2H), 1.40 (s, 9H).

### Example 2-1A

### N-[4-(benzyloxy)-3-chlorobenzyl]-1,2-diaminoethane trityl-resin

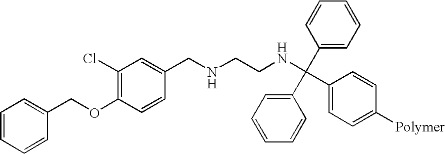

4 g (4.8 mmol) 1,2-diaminoethane trityl-resin are suspended in a mixture of methanol and dichloromethane. 3.55 g (14.4 mmol) 4-benzyloxy-3-chlorobenzaldehyde and 2.6 ml (24 mmol) trimethylorthoformat are added and the mixture is agitated for 2 h at rt, before filtrated. The resin is washed 2 times with dichloromethane, suspended in 20 ml dichloromethane and 8 ml methanol. 907.9 mg (24 mmol) sodium borohydride are added in three portions and the mixture is vigorously agitated for 5 h at rt, before filtrated. The resin is washed successively with: DMF/methanol 1:1 (2 times), methanol/water 1:1, methanol (2 times) and dichloromethane (4 times). The resin can be used without further purification, drying to constant weight can be achieved in high vacuum.

### PREPARATION EXAMPLES

### Example 1

### tert-butyl [2-(benzoyl {4-[(2-fluorobenzyl)oxy]-3-methoxybenzyl}amino)ethyl]carbamate

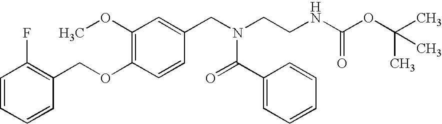

1.00 g (2.47 mmol) tert-butyl [2-({4-[(2-fluorobenzyl)oxy]-3-methoxybenzyl}amino)ethyl]-carbamate is dissolved in 20 ml dichloromethane. 382.3 mg (2.72 mmol) benzoyl chloride and 750.5 mg (7.42 mmol) triethylamine are added at room temperature and the solution is stirred at room temperature for 1.5 hours. A 1N sodium hydroxide solution is then carefully added until basic pH, the organic phase is separated and washed with brine, dried over magnesium sulfate, filtered and concentrated under vacuum to yield the crude desired compound. The crude compound is purified by preparative HPLC (acetonitrile/water) to yield 731 mg (58% of th.) of the title compound.

HPLC (method 1): Rₜ=5.01 min

MS (ESIpos): m/z=509 (M+H)⁺

¹H-NMR (400 MHz, DMSO-d₆): δ=8.10-6.70 (m, 13H), 5.10 (s, 2H), 4.70-4.30 (2s, 2H), 3.75 (m, 3H), 3.30-3.00 (m, 2H), 1.40 (m, 9H)

### Example 2

### tert-butyl {2-[{4-[(2-fluorobenzyl)oxy]-3-methoxybenzyl}(2-thienylcarbonyl)amino]ethyl}-carbamate

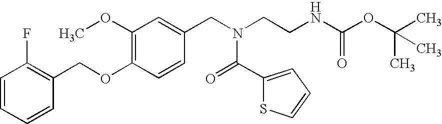

500.0 mg (1.24 mmol) tert-butyl [2-({4-[(2-fluorobenzyl)oxy]-3-methoxybenzyl}amino)ethyl]-carbamate is dissolved in 35 ml dichloromethane. 199.3 mg (1.36 mmol) thiophene-2-carbonyl chloride and 375.3 mg (3.71 mmol) triethylamine are added at room temperature and the solution is stirred at room temperature for 2 hours. The reaction is quenched with 1N hydrochloric acid, the organic phase is separated and washed with a 1N sodium hydroxide solution and with brine, dried over magnesium sulfate, filtered and concentrated under vacuum to yield the crude desired compound. The crude compound is purified by preparative HPLC (acetonitrile/water) to yield 498 mg (78% of th.) of the title compound.

HPLC (method 1): Rₜ=5.09 min

MS (ESIpos): m/z=515 (M+H)⁺

¹H-NMR (300 MHz, DMSO-d₆): δ=7.80-6.70 (m, 11H), 5.05 (s, 2H), 4.70 (s, 2H), 3.75 (s, 3H), 3.40 (m, 2H), 3.20 (m, 2H), 1.40 (s, 9H)

### Example 3

### tert-butyl {2-[{4-[(2,4-difluorobenzyl)oxy]-3-methoxybenzyl}(2-thienylcarbonyl)amino]ethyl}carbamate

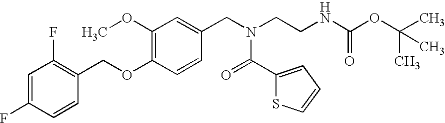

150.0 mg (0.35 mmol) tert-butyl [2-({4-[(2,4-fluorobenzyl)oxy]-3-methoxybenzyl}amino)-ethyl]carbamate is dissolved in 10 ml dichloromethane. 57.3 mg (0.39 mmol) thiophene-2-carbonyl chloride and 107.8 mg (1.06 mmol) triethylamine are added at room temperature and the solution is stirred at room temperature overnight. The reaction is quenched with 1N hydrochloric acid, the organic phase is separated and washed with a 1N sodium hydroxide solution and with brine, dried over magnesium sulfate, filtered and concentrated under vacuum to yield the crude desired compound. The crude compound is purified by preparative HPLC (acetonitrile/water) to yield 75 mg (39% of th.) of the title compound.

LC/MS (method 3): Rₜ=2.82 min,

MS (ESIpos): m/z=532 (M+H)⁺

Using an analogous procedure the following compounds are prepared:

| Example | Starting materials | Structure | Characterisation |
|---|---|---|---|
| 4 | 8A and 3-methylthiophene-2-carbonyl chloride | 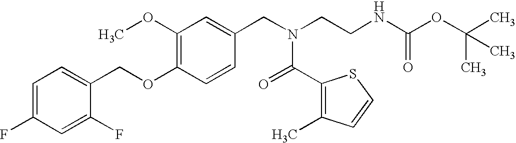 | ¹H-NMR (300 MHz,DMSO-d₆): δ = 7.60-6.70 (m, 9 H), 5.05 (s,2 H), 4.55 (s, 2 H),3.75 (s, 3 H), 3.60 (m,2 H), 3.15 (m, 2 H),2.20 (s, 3 H), 1.40 (s,9 H) |
| 5 | 8A and 3,3-dimethylbutanoylchloride | 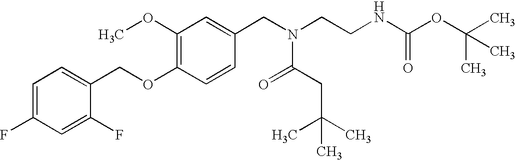 | ¹H-NMR (300 MHz,DMSO-d₆): δ = 7.60-6.70 (m, 7 H), 5.05 (m,2 H), 4.50 (m, 2 H),3.70 (m, 3 H), 3.50 (m,2 H), 3.05 (m, 2 H),2.30 (m, 2 H), 1.40 (m,9 H), 1.00 (m, 9 H) |
| 6 | 7A and thiophene-2-carbonyl chloride | 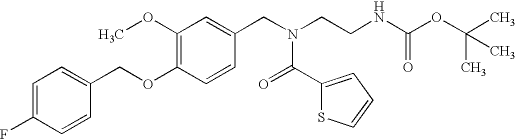 | LC/MS (method 2):Rₜ = 2.87 min,MS (ESIpos):m/z = 515 (M + H)⁺ |
| 7 | 9A and thiophene-2-carbonyl chloride | 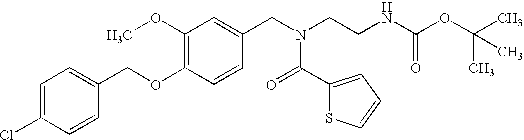 | LC/MS (method 3):Rₜ = 2.92 min,MS (ESIpos):m/z = 531 (M + H)⁺ |
| 8 | 8A and cyclo-hexanecarbonylchloride | 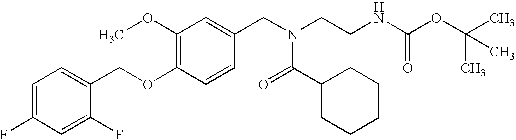 | ¹H-NMR (300 MHz,DMSO-d₆): δ = 7.60(m, 1 H), 7.30-6.70(m, 6 H), 5.05 (m,2 H), 4.50 (m, 2 H),3.75 (m, 3 H), 3.25 (m,2 H), 3.10 (m, 2 H),2.70 (m, 1 H), 1.80-1.10 (m, 19 H) |
| 9 | 8A and 1,2,3,4-tetrahydronaph-thalene-1-carbonylchloride | 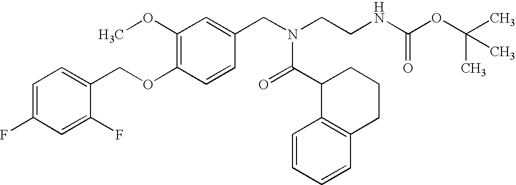 | LC/MS (method 4):Rₜ = 2.87 min,MS (ESIpos):m/z = 581 (M + H)⁺ |
| 10 | 6A and thiophene-2-carbonyl chloride | 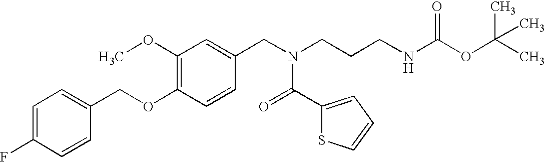 | ¹H-NMR (400 MHz,DMSO-d₆): δ = 7.80-6.70 (m, 11 H), 5.00(s, 2 H), 4.65 (m, 2 H)3.75 (s, 3 H), 3.35 (m,2 H), 2.90 (m, 2 H),1.75 (m, 2 H), 1.35 (s,9 H) |
| 11 | 5A and 4-methyl-benzoyl chloride | 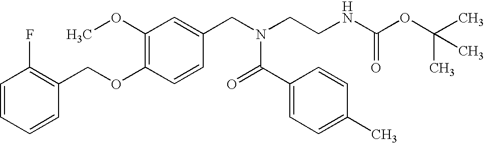 | LC/MS (method 3):Rₜ = 2.89 min,MS (ESIpos):m/z 523 (M + H)⁺ |
| 12 | 8A and butanoylchloride | 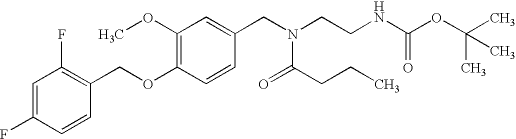 | ¹H-NMR (300 MHz,DMSO-d₆): δ = 7.60-6.60 (m, 7 H), 5.05 (m,2 H), 4.50 (m, 2 H),3.70 (m, 3 H), 3.40 (m,2 H), 3.05 (m, 2 H),2.40 (m, 2 H), 1.60 (m,2 H), 1.40 (s, 9 H),0.90 (m, 3 H) |
| 13 | 8A and 3-methyl-butanoyl chloride | 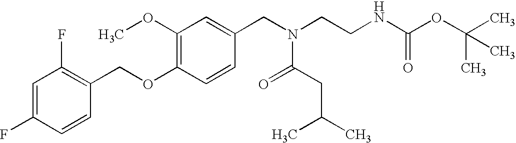 | ¹H-NMR (300 MHz,DMSO-d₆): δ = 7.60-6.70 (m, 7 H), 5.00 (m,2 H), 4.50 (m, 2 H),3.75 (m, 3 H), 3.40 (m,2 H), 3.05 (m, 2 H),2.30-2.00 (m, 3 H),1.40 (m, 9 H), 0.90 (m,6 H) |
| 14 | 8A and benzoylchloride | 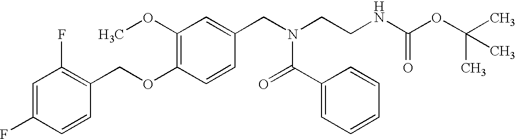 | LC/MS (method 3):Rₜ = 2.81 min,MS (ESIpos):m/z = 527 (M + H)⁺ |

### Example 15

### N-(2-aminoethyl)-N-{4-[(2-fluorobenzyl)oxy]-3-methoxybenzyl}benzamide trifluoroacetate

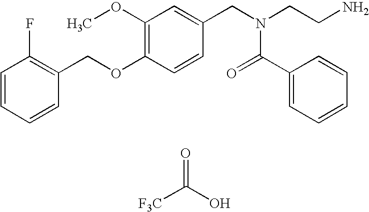

700 mg (1.37 mmol) tert-butyl [2-(benzoyl{4-[(2-fluorobenzyl)oxy]-3-methoxybenzyl}amino)-ethyl]carbamate are dissolved in 90 ml dichloromethane, cooled to 0° C. and 100 ml trifluoroacetic acid are added dropwise. The solution is stirred for 1 h at 0° C. and 2 h at room temperature. The solvent is evaporated under reduced pressure at a maximal temperature of 40° C. The residue is suspended in petroleum ether, the supernatant is separated, diethyl ether is added and the solid is filtered and dried to yield 590 mg (82% of th.) of the title compound.

HPLC (method 5): Rₜ=4.27 min

MS (ESIpos): m/z=409 (M−CF₃COOH+H)⁺

¹H-NMR (400 MHz, DMSO-d₆): δ=7.80 (bs, 3H), 7.60-6.70 (m, 12H), 5.10 (s, 2H), 4.40 (s, 2H), 3.75 (s, 3H), 3.60 (m, 2H), 3.00 (m, 2H)

### Example 16

### N-(2-aminoethyl)-N-{4-[(2,4-difluorobenzyl)oxy]-3-methoxybenzyl}thiophene-2-carboxamide trifluoroacetate

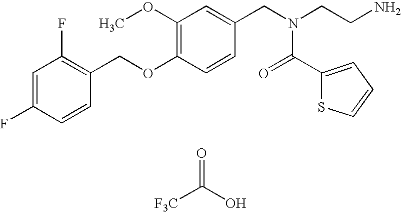

75.0 mg (0.14 mmol) tert-butyl {2-[{4-[(2,4-difluorobenzyl)oxy]-3-methoxybenzyl}(2-thienyl-carbonyl)amino]ethyl}carbamate are dissolved in 20 ml dichloromethane, cooled to 0° C. and 15 ml trifluoroacetic acid are added dropwise. The solution is stirred for 1 h at 0° C. and 2 h at room temperature. The solvent is evaporated under reduced pressure at a maximal temperature of 40° C. The residue is suspended in diethyl ether, the supernatant is separated, diethyl ether is added and the solid is filtered and dried to yield 26 mg (34% of th.) of the title compound.

HPLC (method 5): Rₜ=4.38 min

MS (ESIpos): m/z=433 (M−CF₃COOH+H)⁺

¹H-NMR (400 MHz, DMSO-d₆): δ=7.80 (m, 4H), 7.60-6.70 (m, 8H), 5.05 (s, 2H), 4.75 (s, 2H), 3.75 (s, 3H), 3.60 (m, 2H), 3.10 (m, 2H)

### Example 17

### N-(2-aminoethyl)-N-{4-[(2-fluorobenzyl)oxy]-3-methoxybenzyl}thiophene-2-carboxamide trifluoroacetate

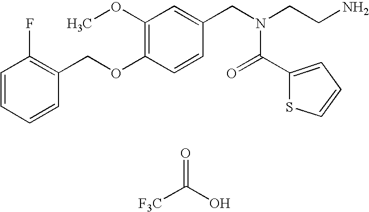

100 mg (0.194 mmol) tert-butyl {2-[{4-[(2-fluorobenzyl)oxy]-3-methoxybenzyl}(2-thienyl-carbonyl)amino]ethyl}carbamate are dissolved in 20 ml dichloromethane, cooled to 0° C. and 10 ml trifluoroacetic acid are added dropwise. The solution is stirred for 1 h at 0° C. and 2 h at room temperature. The solvent is evaporated under reduced pressure at a maximal temperature of 40° C. The residue is suspended in diethyl ether, the supernatant is separated, diethyl ether is added and the solid is filtered and dried to yield 65 mg (63% of th.) of the title compound.

HPLC (method 5): Rₜ=4.28 min

MS (ESIpos): m/z=415 (M−CF₃COOH+H)⁺

¹H-NMR (400 MHz, DMSO-d₆): δ=7.80 (m, 4H), 7.60-6.70 (m, 9H), 5.10 (s, 2H), 4.70 (s, 2H), 3.75 (s, 3H), 3.60 (m, 2H), 3.05 (m, 2H)

### Example 18

### N-(3-aminopropyl)-N-{4-[(4-fluorobenzyl)oxy]-3-methoxybenzyl}benzamide trifluoroacetate

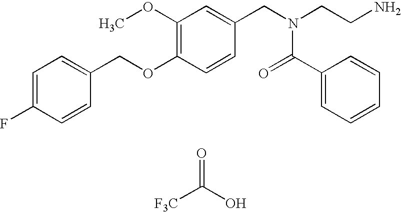

60 mg (0.11 mmol) tert-butyl [2-(benzoyl{4-[(4-fluorobenzyl)oxy]-3-methoxybenzyl}amino)-propyl]carbamate are dissolved in 10 ml dichloromethane, cooled to 0° C. and 7 ml trifluoroacetic acid are added dropwise. The solution is stirred for 1 h at 0° C. and 2 h at room temperature. The solvent is evaporated under reduced pressure at a maximal temperature of 40° C. The crude residue is purified by preparative HPLC (acetonitrile/water) to yield 24 mg (38% of th.) of the title compound.

HPLC (method 5): Rₜ=4.40 min

MS (ESIpos): m/z=423 (M−CF₃COOH+H)⁺

¹H-NMR (400 MHz, DMSO-d₆): δ=7.70 (bs, 3H), 7.60-6.70 (m, 12H), 5.05 (s, 2H), 4.50 (m, 2H), 3.75 (s, 3H), 3.40 (m, 2H), 2.80 (m, 2H), 1.80 (m, 2H)

Using an analogous procedure the following compounds are prepared:

| Example | Starting materials | Structure | Characterisation |
|---|---|---|---|
| 19 | Example 4 | 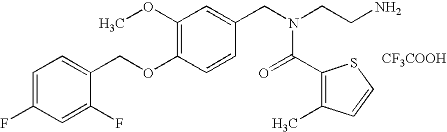 | ¹H-NMR (400 MHz,DMSO-d₆): δ = 7.80(m, 3 H), 7.60-7.50 (m,2 H), 7.30-7.00 (m, 4 H),6.80-6.70 (m, 2 H), 5.05(s, 2 H), 4.55 (s, 2 H),3.75 (s, 3 H), 3.60 (m,2 H), 2.95 (m, 2 H), 2.20(s, 3 H) |
| 20 | Example 5 | 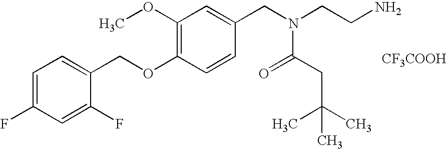 | ¹H-NMR (300 MHz,DMSO-d₆): δ = 7.80-7.50 (m, 4 H), 7.35-7.00(m, 3 H), 6.90-6.55 (m,2 H), 5.05 (m, 2 H), 4.50(m, 2 H), 3.75 (m, 3 H),3.50 (m, 2 H), 3.00-2.85(m, 2 H), 2.30 (s, 2 H),1.00 (m, 9 H) |
| 21 | Example 6 | 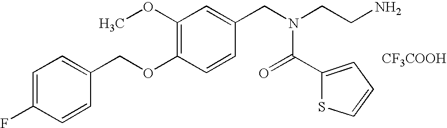 | ¹H-NMR (300 MHz,DMSO-d₆): δ = 7.80(m, 4 H), 7.50-6.70 (m,9 H), 5.05 (s, 2 H), 4.70(s, 2 H), 3.75 (s, 3 H),3.60 (m, 2 H), 3.05 (m,2 H) |
| 22 | Example 7 | 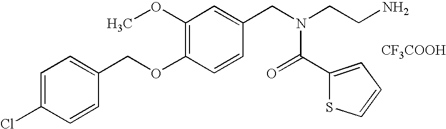 | ¹H-NMR (300 MHz,CDCl₃): δ = 8.50 (bs,3 H), 7.50-6.70 (m,10 H), 5.10 (s, 2 H), 4.75(s, 2 H), 3.75 (s, 3 H),3.70 (m, 2 H), 3.20 (m,2 H) |
| 23 | Example 8 | 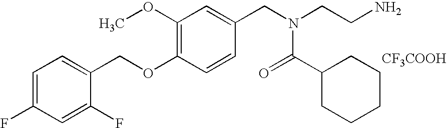 | ¹H-NMR (300 MHz,DMSO-d₆): δ = 7.80-7.50 (m, 4 H), 7.40-7.00(m, 3 H), 6.80-6.65 (m,2 H), 5.05 (m, 2 H), 4.50(m, 2 H), 3.75 (m, 3 H),3.50 (m, 2 H), 3.00-2.80(m, 2 H), 2.60 (m, 1 H),1.80-1.10 (m, 10 H) |
| 24 | Example 9 | 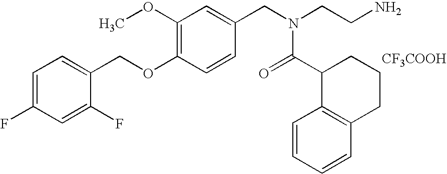 | ¹H-NMR (300 MHz,DMSO-d₆): δ = 7.80-7.50 (m, 4 H), 7.30-7.00(m, 6 H), 6.90-6.70 (m,3 H), 5.05 (m, 2 H),4.70-4.50 (m, 2 H), 4.20(m, 1 H), 3.75 (m, 3 H),3.50 (m, 2 H), 3.10-2.70(m, 4 H), 2.10-1.70 (m,4 H) |
| 25 | Example 10 | 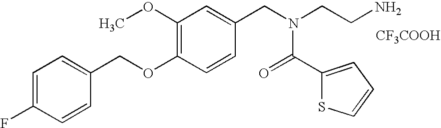 | ¹H-NMR (400 MHz,DMSO-d₆): δ = 7.80-7.60 (m, 4 H), 7.50-6.70(m, 9 H), 5.05 (s, 2 H),4.70 (m, 2 H), 3.75 (s,3 H), 3.45 (m, 2 H), 2.80(m, 2 H), 1.85 (m, 2 H) |
| 26 | Example 11 | 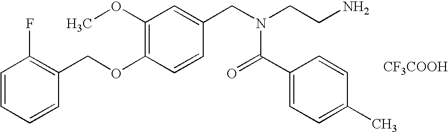 | ¹H-NMR (300 MHz,DMSO-d₆): δ = 7.80(bs, 3 H), 7.60-6.70 (m,11 H), 5.10 (s, 2 H), 4.50(s, 2 H), 3.75 (s, 3 H),3.55 (s, 2 H), 3.00 (s,2 H), 2.35 (s, 3 H) |
| 27 | Example 12 | 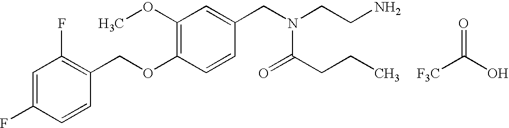 | ¹H-NMR (300 MHz,DMSO-d₆): δ = 7.80-7.50 (m, 4 H), 7.35-7.00(m, 3 H), 6.90-6.65 (m,2 H), 5.05 (m, 2 H), 4.50(m, 2 H), 3.75 (m, 3 H),3.40 (m, 2 H), 2.90 (m,2 H), 2.40 (m, 2 H), 1.60(m, 2 H), 0.90 (m, 3 H) |
| 28 | Example 13 | 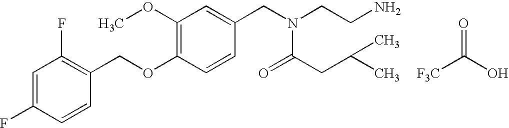 | ¹H-NMR (300 MHz,DMSO-d₆): δ = 7.80-7.50 (m, 4 H), 7.35-7.00(m, 3 H), 6.90-6.65 (m,2 H), 5.05 (m, 2 H), 4.50(m, 2 H), 3.75 (m, 3 H),3.40 (m, 2 H), 2.90 (m,2 H), 2.30 (m, 2 H), 2.05(m, 1 H), 0.90 (m, 6 H) |
| 29 | Example 14 | 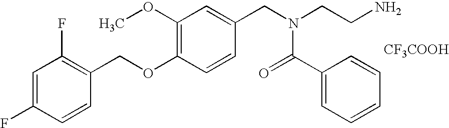 | ¹H-NMR (400 MHz,DMSO-d₆): δ = 7.80-6.65 (m, 14 H), 5.05 (s,2 H), 4.50 (m, 2 H), 3.75(s, 3 H), 3.55 (m, 2 H),3.00 (m, 2 H) |

### Example 30

### N-(2-aminoethyl)-N-{4-[(2,4-difluorobenzyl)oxy]-3-methoxybenzyl}cyclopropanecarboxamide

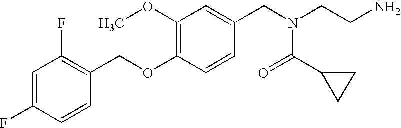

39 mg (0.1 mmol) tert-butyl [2-({4-[(2,4-fluorobenzyl)oxy]-3-methoxybenzyl}amino)ethyl]carbamate are dissolved in 60 ml dichloroethane. 10.4 mg (0.1 mmol) cyclopropanecarbonyl chloride and 13.0 mg (0.13 mmol) triethylamine are added at room temperature and the solution is stirred at room temperature during 5 hours. The solvent is evaporated under vacuum and 0.2 ml trifluoroacetic acid are added. The solution is stirred for 30 min at room temperature, 0.5 ml dimethylsulfoxide are added. The solution is filtered and the crude compound is purified by preparative HPLC (acetonitrile/water) to yield the title compound.

LC-MS (method 6): Rₜ=1.53 min,

MS (ESIpos): m/z=391 (M+H)⁺

Using an analogous procedure the following compounds are prepared:

| Example | Starting materials | Structure | Characterisation |
|---|---|---|---|
| 31 | 8A and 1-methyl-3-(trifluoromethyl)-1H-pyrazole-4-carbonyl chloride | 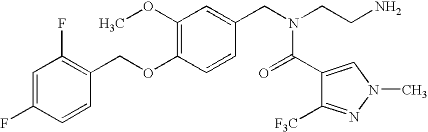 | LC-MS (method 6):Rₜ = 1.57 min,MS (ESIpos):m/z = 499 (M + H)⁺ |
| 32 | 8A and (2E)-but-2-enoyl chloride | 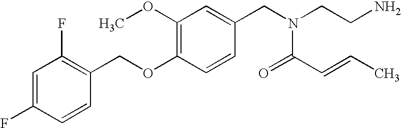 | LC-MS (method 6):Rₜ = 1.52 min,MS (ESIpos):m/z = 391 (M + H)⁺ |
| 33 | 8A and 2-methyl-cyclopropane-carbonyl chloride | 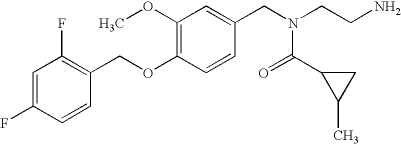 | LC-MS (method 6):Rₜ = 1.58 min,MS (ESIpos):m/z = 405 (M + H)⁺ |
| 34 | 8A and 2-thienyl-acetyl chloride | 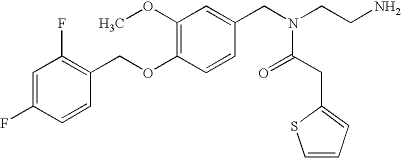 | LC-MS (method 6):Rₜ = 1.58 min,MS (ESIpos):m/z = 447 (M + H)⁺ |
| 35 | 8A and 2-thienyl-acetyl chloride | 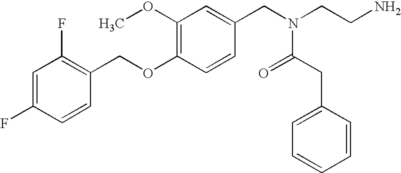 | LC-MS (method 6):Rₜ = 1.55 min,MS (ESIpos):m/z = 441 (M + H)⁺ |
| 36 | 8A and 2-methyl-butanoyl chloride | 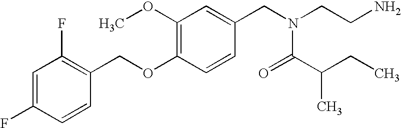 | LC-MS (method 6):Rₜ = 1.54 min,MS (ESIpos):m/z = 407 (M + H)⁺ |
| 37 | 8A and 3-methyl-benzoyl chloride | 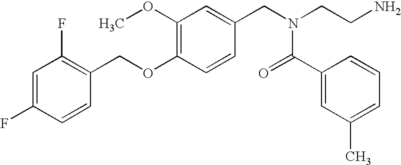 | LC-MS (method 6):Rₜ = 1.65 min,MS (ESIpos):m/z = 441 (M + H)⁺ |

### Example 38

### N-benzyl-N-{4-[(4-fluorobenzyl)oxy]-3-methoxybenzyl}benzamide

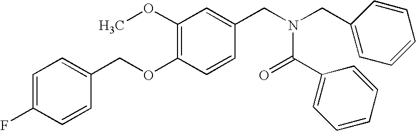

40 mg (0.114 mmol) N-benzyl-1-{4-[(4-fluorobenzyl)oxy]-3-methoxyphenyl}methanamine is dissolved in 5 ml dichloromethane. 17.6 mg (0.125 mmol) benzoyl chloride and 17.3 mg (0.17 mmol) triethylamine are added at room temperature and the solution is stirred at room temperature for 2 hours. A 1N sodium hydroxide solution is then carefully added until basic pH, the organic phase is separated and washed with brine, dried over magnesium sulfate, filtered and concentrated under vacuum to yield the crude desired compound. The crude compound is purified by preparative HPLC (acetonitrile/water) to yield 40.8 mg (79% of th.) of the title compound.

HPLC (method 5): Rₜ=5.35 min

MS (ESIpos): m/z=456 (M+H)⁺

¹H-NMR (300 MHz, DMSO-d₆): δ=7.50-7.05 (m, 14H), 7.00 (d, 1H), 6.90-6.55 (m, 2H), 5.00 (s, 2H), 4.60-4.20 (m, 4H), 3.70 (m, 3H)

### Example 39

### N-benzyl-N-{4-[(4-fluorobenzyl)oxy]-3-methoxybenzyl}thiophene-2-carboxamide

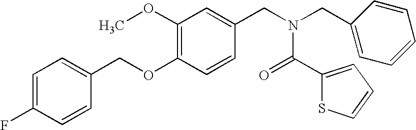

40 mg (0.114 mmol) N-benzyl-1-{4-[(4-fluorobenzyl)oxy]-3-methoxyphenyl}methanamine is dissolved in 5 ml dichloromethane. 18.4 mg (0.125 mmol) thiophene-2-carbonyl chloride and 17.3 mg (0.17 mmol) triethylamine are added at room temperature and the solution is stirred at room temperature for 2 hours. A 1N sodium hydroxide solution is then carefully added until basic pH, the organic phase is separated and washed with brine, dried over magnesium sulfate, filtered and concentrated under vacuum to yield the crude desired compound. The crude compound is purified by preparative HPLC (acetonitrile/water) to yield 37.1 mg (71% of th.) of the title compound.

HPLC (method 5): Rₜ=5.35 min

MS (ESIpos): m/z=462 (M+H)⁺

¹H-NMR (300 MHz, DMSO-d₆): δ=7.75 (d, 1H), 7.50-7.00 (m, 12H), 6.90-6.70 (m, 2H), 5.00 (s, 2H), 4.70 (m, 4H), 3.70 (s, 3H)

Using an analogous procedure the following compounds are prepared:

| Example | Starting materials | Structure | Characterisation |
|---|---|---|---|
| 40 | 10A and benzoylchloride | 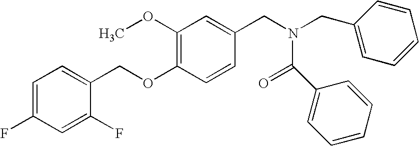 | ¹H-NMR (300 MHz,DMSO-d₆): δ = 7.65-7.00 (m, 14 H), 6.90-6.55 (m, 2 H), 5.00 (s,2 H), 4.60-4.30 (m, 4 H),3.70 (m, 3 H) |
| 41 | 12A and benzoylchloride | 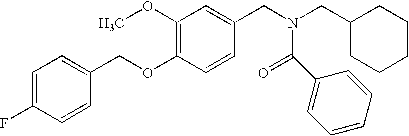 | ¹H-NMR (300 MHz,DMSO-d₆): δ = 7.50-7.10 (m, 9H), 7.00-6.80(m, 2 H), 6.60 (m, 1 H),5.00 (s, 2 H), 4.60-4.20(m, 2 H), 3.70 (m, 3 H),3.30-2.90 (m, 2 H),(m, 2 H),1.80-0.80 (m, 10 H),0.50 (m, 1 H) |
| 42 | 12A and thiophene-2-carbonyl chloride | 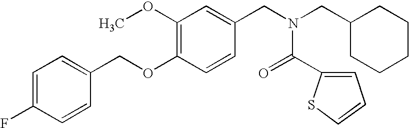 | ¹H-NMR (300 MHz,DMSO-d₆): δ = 7.70 (d,1 H), 7.50-7.00 (m, 7 H),6.80-6.65 (m, 2 H), 5.00(s, 2 H), 4.70 (m, 2 H),3.70 (s, 3 H), 3.30-3.10(m, 2 H), 1.80-0.80 (m,11 H) |
| 43 | 11A and thiophene-2-carbonyl chloride | 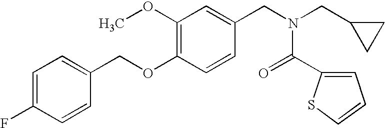 | ¹H-NMR (300 MHz,DMSO-d₆): δ = 7.70 (d,1 H), 7.50-7.00 (m, 7 H),6.90 (s, 1 H), 6.75 (d,1 H), 5.00 (s, 2 H), 4.75(m, 2 H), 3.70 (s, 3 H),1.05 (m, 1 H), 0.95 (m,2 H), 0.20 (m, 2 H) |

Using an analogous procedure as for example 3 the following compounds are prepared:

| example | starting materials | structure | analytical data |
|---|---|---|---|
| 44 | 9A and benzoylchloride | 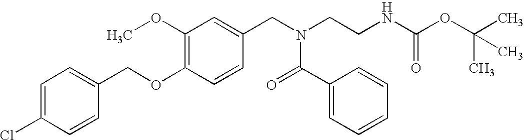 | LC-MS (method 2): Rₜ =2.90 min, m/z = 525(M + H)⁺ |
| 45 | 9A and 4-metyl-benzoyl chloride | 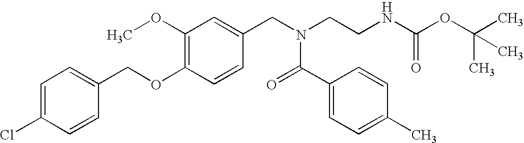 | LC-MS (method 4): Rₜ =3.02 min, m/z = 539(M + H)⁺ |
| 46 | 8A and 4-fluoro-benzoyl chloride | 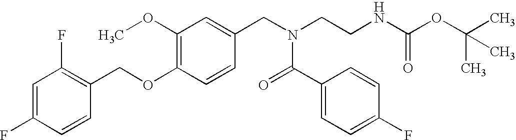 | ¹H-NMR (400 MHz,DMSO-d₆): δ = 7.60(m, 1 H), 7.48 (m, 2 H),7.34-7.22 (m, 3 H), 7.14(m, 1 H), 7.05 (m, 1 H),6.97 (m, 1 H), 6.87 (m,1 H), 6.68 (m, 1 H), 5.05(s, 2 H), 4.62 (m, 1 H),4.41 (m, 1 H), 3.74 (s,3 H), 3.17 (m, 2 H), 3.02(m, 2 H), 1.36 (s, 9 H) |
| 47 | 8A and 2-fluoro-benzoyl chloride | 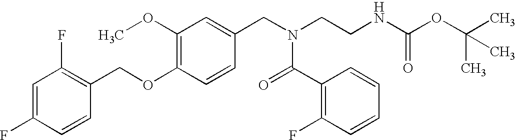 | HPLC (method 5): Rₜ =5.06 min; MS (ESI+)m/z = 545 (M + H)⁺ |
| 48 | 6A and 4-chloro-benzoyl chloride | 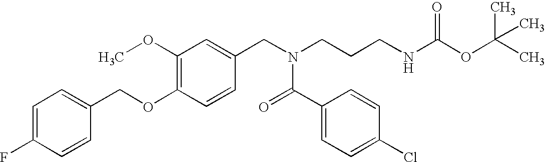 | ¹H-NMR (400 MHz,DMSO-d₆): δ = 7.55-7.38 (m, 6 H), 7.22 (t,2 H), 7.01 (d, 1 H), 6.95(m, 1 H), 6.82 (m, 1 H),6.68 (m, 1 H), 5.05 (s,2 H), 4.59 (m, 1 H), 4.38(m, 1 H), 3.74 (m, 3 H),3.12-2.85 (m, 2 H), 2.74(m, 2 H), 1.75-1.50 (m,2 H), 1.42-1.21 (m, 9 H), |
| 49 | 7A and benzoylchloride | 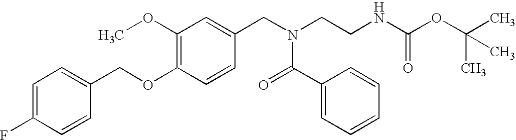 | ¹H-NMR (400 MHz,DMSO-d₆): δ = 7.56-7.31 (m, 6 H), 7.23 (t,2 H), 7.07-6.78 (m, 3 H),6.66 (m, 1 H), 5.05 (s,2 H), 4.64 (m, 1 H), 4.38(m, 1 H), 3.82-3.65 (m,3 H), 3.27-2.92 (m, 4 H),1.37 (m, 9H). |

Using an analogous procedure as for example 18 the following compounds are prepared:

| example | starting materials | structure | analytical data |
|---|---|---|---|
| 50 | example 44 | 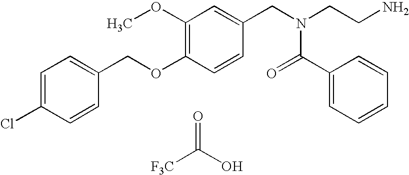 | ¹H-NMR (400 MHz,DMSO-d₆): δ = 7.78 (m,2 H), 7.70-7.59 (m, 1 H),7.56-7.37 (m, 7 H), 7.01 (d,1 H), 6.81 (m, 1 H), 5.05 (s,2 H), 4.44 (s, 2 H), 3.75 (s,3 H), 3.55 (m, 2 H), 3.03(m, 2 H). |
| 51 | example 45 | 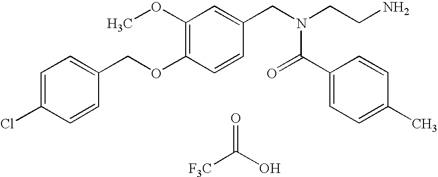 | ¹H-NMR (400 MHz,DMSO-d₆): δ = 7.89-7.68(m, 2 H), 7.50-7.33 (m,5 H), 7.26 (m, 2 H), 7.01 (d,1 H), 6.81 (m, 1 H), 5.05 (s,2 H), 4.43 (s, 2 H), 3.75 (s,3 H), 3.55 (m, 2 H), 3.01(m, 2 H), 2.33 (s, 3 H). |
| 52 | example 46 | 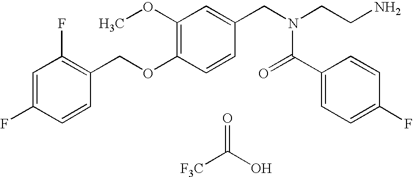 | ¹H-NMR (300 MHz,DMSO-d₆): δ = 7.72 (m,1 H), 7.65-7.50 (m, 3 H),7.37-7.24 (m, 3 H), 7.14(m, 2 H), 7.08 (d, 1 H), 6.81(m, 1 H), 5.05 (s, 2 H), 4.43(m, 2 H), 3.74 (s, 3 H), 3.55(m, 2 H), 3.01 (m, 2 H). |
| 53 | example 47 | 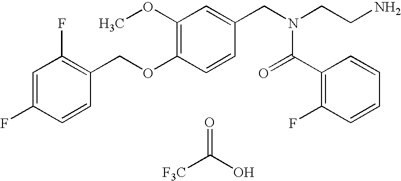 | ¹H-NMR (300 MHz,DMSO-d₆): δ = 7.78 (m,1 H), 7.65-7.45 (m, 3 H),7.39-7.23 (m, 3 H), 7.18-7.03 (m, 2 H), 6.81 (m,1 H), 5.05 (s, 2 H), 4.47 (s,2 H), 3.72 (s, 3 H), 3.59 (m,2 H), 2.98 (m, 2 H). |

### Example 54

### N-(3-aminopropyl)-4-chloro-N-{4-[(4-fluorobenzyl)oxy]-3-methoxybenzyl}benzamide hydrochloride

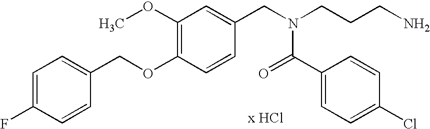

170 mg (0.305 mmol) tert-butyl [3-((4-chlorobenzoyl){4-[(4-fluorobenzyl)oxy]-3-methoxy-benzyl}amino)propyl]carbamate are dissolved in 2 ml dioxane and treated at rt with 1 ml 4N solution of hydrochloric acid in dioxane. The mixture is stirred over night, before the solvents are removed in vacuo. The residue is treated with petroleum ether, the supernatant layer is decanted. Again to the residual oil diethyl ether is added, again the supernatant layer is decanted. The residual oil is purified by preparative reverse phase HPLC to afford 21.4 mg (14% of th.) of the title compound.

HPLC (method 5): Rₜ=4.43 min; MS (ESIpos): m/z=457 (M−HCl+H)⁺

¹H-NMR (400 MHz, DMSO-d₆): δ=7.78 (m, 3H), 7.58-7.41 (m, 5H), 7.22 (t, 2H), 7.01 (d, 1H), 6.69 (m, 1H), 5.05 (s, 2H), 4.61/4.39 (2m, 2H) 3.72 (s, 3H), 3.42/3.15 (2m, 2H), 2.84/2.58 (2m, 2H), 1.95-1.65 (m, 2H).

### Example 55

### N-(2-aminoethyl)-N-{4-[(4-fluorobenzyl)oxy]-3-methoxybenzyl}thiophene-2-carboxamide

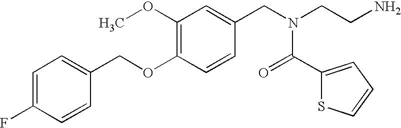

70 mg (0.136 mmol) tert-butyl {2-[{4-[(4-fluorobenzyl)oxy]-3-methoxybenzyl}(2-thienyl-carbonyl)amino]ethyl}carbamate are dissolved in 2 ml dioxane and treated at rt with 1 ml 4N solution of hydrochloric acid in dioxane. The solvents are removed in vacuo and the residue is treated with diethyl ether. The resulting supernatant layer is decanted. The residual oil is dissolved, washed with 1N sodium hydroxide solution and purified after evaporation by preparative reverse phase HPLC to afford 4.3 mg (7% of th.) of the title compound.

HPLC (method 5): Rₜ=5.03 min; MS (ESIpos): m/z=659 (M+H)⁺

¹H-NMR (300 MHz, DMSO-d₆): δ=7.75 (d, 1H), 7.53-7.43 (m, 2H), 7.28-7.15 (m, 2H), 7.08 (t, 1H), 7.01 (d, 1H), 6.95 (s, 1H), 6.85 (m, 1H), 6.79-6.71 (m, 1H), 5.03 (d, 2H), 4.69 (m, 2H) 3.72 (d, 3H), 3.51 (m, 2H), 2.74 (m, 2H).

### Example 56

### N-(2-aminoethyl)-N-{4-[(4-fluorobenzyl)oxy]-3-methoxybenzyl}benzamide

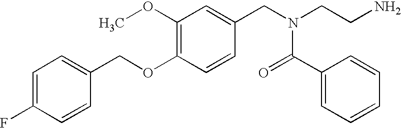

80 mg (0.157 mmol) tert-butyl [2-(benzoyl{4-[(4-fluorobenzyl)oxy]-3-methoxybenzyl}amino)-ethyl]carbamate are dissolved in 2 ml dioxane and treated at rt with 1 ml 4N solution of hydrochloric acid in dioxane. The solvents are removed in vacuo and the residue is treated with diethyl ether. The resulting supernatant layer is decanted. The residual oil is dissolved, washed with 1N sodium hydroxide solution and purified after evaporation by preparative reverse phase HPLC to afford 10.2 mg (15% of th.) of the title compound.

HPLC (method 5): Rₜ=4.37 min; MS (ESIpos): m/z=409 (M+H)⁺

¹H-NMR (300 MHz, DMSO-d₆): δ=7.84 (dd, 1H), 7.53-7.38 (m, 5H), 7.28-7.15 (m, 2H), 7.04-7.91 (m, 2H), 6.81 (dd, 1H), 6.71 (m, 1H), 5.02 (d, 2H), 4.64/4.42 (2m, 2H) 3.81-3.69 (m, 4H therein: 3.72 (s, 3H)), 2.93 (m, 1H), 2.68 (t, 2H).

Using an analogous procedure as for example 30 the following compounds are prepared:

| example | starting materials | structure | analytical data |
|---|---|---|---|
| 57 | 8A and 3,3-dimethyl-butyricacid chloride | 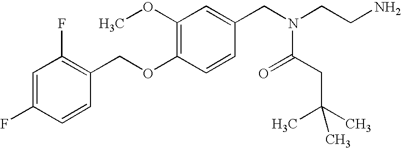 | LC-MS (method 6):Rₜ = 1.61 min,MS (ESIpos):m/z = 421 (M + H)⁺ |
| 58 | 8A and thiophene-carboxylic acidchloride | 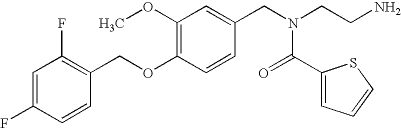 | LC-MS (method 6):Rₜ = 1.56 min,MS (ESIpos):m/z = 433 (M + H)⁺ |
| 59 | 8A and 3-methyl-thiophenecarboxylicacid chloride | 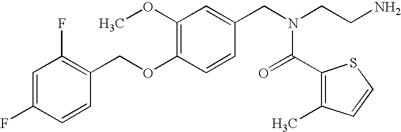 | LC-MS (method 6):Rₜ = 1.58 min,MS (ESIpos):m/z = 447 (M + H)⁺ |

### Example 1-1

### tert-butyl {2-[[4-(benzyloxy)-3-methoxybenzyl](2-thienylcarbonyl)amino]ethyl}carbamate

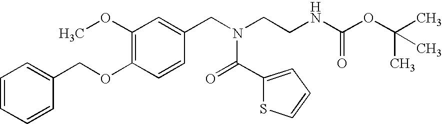

1.60 g (4.14 mmol) tert-butyl (2-{[4-(benzyloxy)-3-methoxybenzyl]amino}ethyl)carbamate are dissolved in 20 ml dichloromethane. 0.67 g (4.55 mmol) thiophene-2-carbonyl chloride and 0.63 g (6.21 mmol) triethylamine are added at room temperature and the solution is stirred at this temperature overnight. The reaction is quenched with water, the organic phase is separated and washed with saturated sodium bicarbonate solution and with brine, dried over magnesium sulfate, filtered and concentrated under vacuum to yield the crude desired compound. The crude is purified by preparative HPLC (acetonitrile/water) to yield 1.11 g (54% of th.) of the title compound.

HPLC (method 2-1): Rₜ=5.03 min

MS (ESIpos): m/z=497 (M+H)⁺

¹H-NMR (300 MHz, DMSO-d₆): δ=7.70 (d, 1H), 7.40-7.25 (m, 6H), 7.10-6.70 (m, 5H), 5.05 (s, 2H), 4.70 (s, 2H), 3.75 (s, 3H), 3.40 (m, 2H), 3.20 (m, 2H), 1.40 (s, 9H).

### Example 2-1

### tert-butyl (2-{benzoyl[4-(benzyloxy)-3-methoxybenzyl]amino}ethyl)carbamate

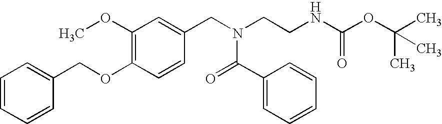

207.5 mg (0.54 mmol) tert-butyl (2-{[4-(benzyloxy)-3-methoxybenzyl]amino}ethyl)carbamate are dissolved in 5 ml dichloromethane. 83.9 mg (0.59 mmol) benzoyl chloride and 81.5 mg (0.81 mmol) triethylamine are added at room temperature and the solution is stirred at this temperature overnight. The reaction is quenched with 1N hydrochloric acid, the organic phase is separated and washed with brine, dried over magnesium sulfate, filtered and concentrated under vacuum to yield the crude desired compound. The crude is purified by preparative HPLC (acetonitrile/water) to yield 160 mg (51% of th.) of the title compound.

HPLC (method 2-1): Rₜ=4.99 min

MS (ESIpos): m/z=491 (M+H)⁺

¹H-NMR (300 MHz, DMSO-d₆): δ=8.20-6.70 (m, 14H), 5.05 (s, 2H), 4.70 (m, 2H), 3.75 (s, 3H), 3.40 (m, 2H), 3.10 (m, 2H), 1.40 (m, 9H).

### Example 3-1

### N-(2-aminoethyl)-N-[4-(benzyloxy)-3-methoxybenzyl]thiophene-2-carboxamide hydrochloride

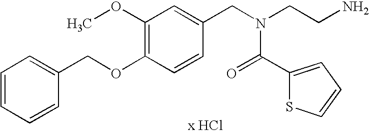

980 mg (1.97 mmol) tert-butyl {2-[[4-(benzyloxy)-3-methoxybenzyl](2-thienylcarbonyl)-amino]ethyl}carbamate are dissolved in 5 ml dioxane and 5 ml of a 4M solution of hydrochloric acid in dioxane are added dropwise. The solution is stirred overnight, 1.5 ml of the 4M hydrochloric acid dioxane solution are added and stirred overnight again. The precipitated solid is filtered, washed with diethyl ether and dried. The title compound is obtained as a solid (744 mg, 87% of th.).

HPLC (method 2-1): Rₜ=4.27 min

MS (ESIpos): m/z=397 (M−HCl+H)⁺

¹H-NMR (400 MHz, DMSO-d₆): δ=7.80 (br.s, 4H), 7.45-7.00 (m, 8H), 6.90-6.70 (m, 2H), 5.05 (s, 2H), 4.70 (s, 2H), 3.75 (s, 3H), 3.60 (s, 2H), 3.00 (s, 2H).

### Example 4-1

### N-(2-aminoethyl)-N-[4-(benzyloxy)-3-methoxybenzyl]benzamide trifluoroacetate

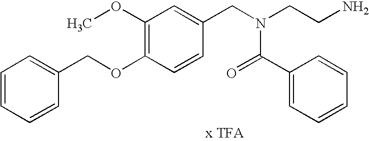

140 mg (0.29 mmol) tert-butyl (2-{benzoyl[4-(benzyloxy)-3-methoxybenzyl]amino}ethyl)carbamate are dissolved in 40 ml dichloromethane and the solution is cooled to 0° C., then 20 ml of trifluoroacetic acid are added dropwise. The solution is stirred 1 hour at this temperature and 2 hours at room temperature. The solution is concentrated under vacuum at 40° C. and the crude product is purified by preparative HPLC (acetonitrile:water) to yield 108 mg (70% of th.) of the title compound.

HPLC (method 2-1): Rₜ=4.22 min

MS (ESIpos): m/z=391 (M−CF₃COOH+H)⁺

¹H-NMR (400 MHz, DMSO-d₆): δ=7.90 (d, 1H), 7.80 (br.s, 3H), 7.60-7.20 (m, 10H), 7.00 (d, 1H), 6.70 (m, 1H), 5.05 (s, 2H), 4.40 (s, 2H), 3.75 (s, 3H), 3.60 (s, 2H), 3.00 (s, 2H).

Using an analogous procedure the following compounds are prepared:

| Example | Starting materials | Structure | Characterisation |
|---|---|---|---|
| 5-1 | 1-1A and cyclohexane-carbonyl chloride | 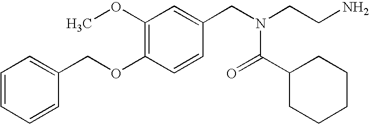 | LC/MS (method 3-1): Rₜ =1.94 min, m/z =397 (M + H)⁺ |
| 6-1 | 1-1A and 3-methylbut-2-enoyl chloride | 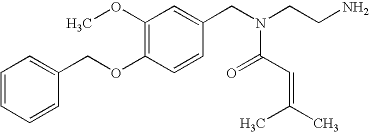 | |
| 7-1 | 1-1A and 3-cyclo-pentyl-propanoylchloride | 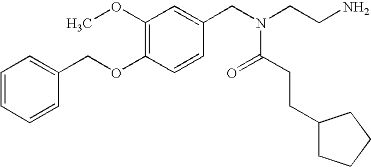 | LC/MS (method 3-1): Rₜ =2.07 min, m/z =411 (M + H)⁺ |

### Example 8-1

### tert-butyl {2-[[4-(benzyloxy)-3-methoxybenzyl](4-fluorobenzoyl)amino]ethyl}carbamate

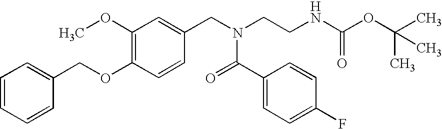

15 g (38.81 mmol) tert-butyl-(2-{[4-(benzyloxy)-3-methoxybenzyl]amino}ethyl)carbamate are dissolved in 75 ml dichloromethane. 13.5 ml (97 mmol) triethylamine are added and the mixture is colled to 0° C. A solution of 6.77 g (42.69 mmol) 4-fluorobenzoylchloride in 25 ml dichloromethane is added dropwise. The resulting mixture is stirred 30 min at 0° C., before poured onto water. The organic layer is washed with water and brine and dried over magnesium chloride. After evaporation of valotiles the oily residue is dissolved in 100 ml ethyl acetate, washed 2 times with buffer solution (pH 7) and brine, dried over magnesium sulfate and concentrated in vacuo. The crude product is purified by chromatography on silica gel (cyclohexane/ethyl acetate 3:1 to 2:1) to yield 18.34 g (93% of th.) of the title compound.

LC-MS (method 4-1): Rₜ=2.69 min, m/z=509 (M+H)⁺

¹H-NMR (300 MHz, DMSO-d₆): δ=7.59-7.20 (m, 9H), 7.11-6.56 (m, 4H), 5.05 (s, 2H), 4.61 (s, 1H), 4.39 (s, 1H), 3.75 (m, 3H), 3.45-2.90 (m, 4H), 1.36 (m, 9H).

### Example 9-1

### tert-butyl(2-{[4-(benzyloxy)-3-methoxybenzyl][(4-fluorophenyl)acetyl]amino}ethyl)carbamate

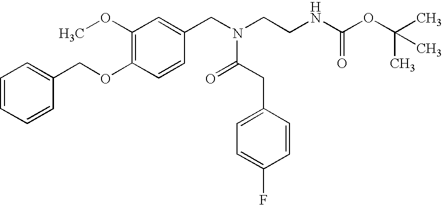

To a solution of 200 mg (5.1 mmol) tert-butyl-(2-{[4-(benzyloxy)-3-methoxybenzyl]amino}-ethyl)carbamate in 2 ml DMF are added at rt 87.4 mg (0.57 mmol) 4-fluorophenylacetic acid, 76.9 mg (0.57 mmol) HOBT, a catalytic amount of 4-DMAP and 0.135 ml (0.77 mmol) N,N-diisopropylethylamine. The mixture is cooled to 0° C. and 109 g (0.57 mmol) 1-(3-dimethylaminopropyl)-3-ethylcarbodiimide hydrochloride are added at once. The reaction mixture is allowed to warm up to rt and stirring is continued over night, before poured onto water and extracted 3 times with ethyl acetate. The combined organic layers are washed with brine, dried over sodium sulfate and concentrated in vacuo. The crude product is purified by chromatography on silica gel (cyclohexane/ethyl acetate 3:1) to yield 225 mg (83% of th.) of the title compound.

LC-MS (method 1-1): Rₜ=2.67 min, m/z=523 (M+H)+

¹H-NMR (300 MHz, DMSO-d₆): δ=(signals indicate presence of rotamere in a 2:1 ratio) 7.48-6.41 (m, 13H), 5.07/5.02 (2s, 2H), 4.55/4.43 (2s, 2H), 3.76/3.71/3.68 (3s, 5H), 3.32-3.19 (m, 2H), 3.16-2.98 (m, 2H), 1.39/1.86 (2s, 9H).

### Example 10-1

### N-(2-aminoethyl)-N-[4-(benzyloxy)-3-methoxybenzyl]-4-fluorobenzamide hydrochloride

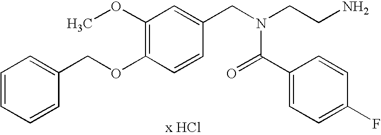

100 mg (0.197 mmol) tert-butyl{2-[[4-(benzyloxy)-3-methoxybenzyl](4-fluorobenzoyl)amino]-ethyl}carbamate are dissolved in 1 ml dioxane and treated at rt with 0.49 ml 4M solution of hydrochloric acid in dioxane. After stirring for 1 h at rt the mixture is dissolved in methanol and purified directly by RP HPLC chromatography (gradient water/acetonitrile) to afford 36.9 mg (42% of th.) of the title compound.

LC-MS (method 4-1): Rₜ=1.78 min, m/z=409 (M−HCl+H)⁺

¹H-NMR (400 MHz, DMSO-d₆): δ=7.98 (bs, 3H), 7.62 (m, 2H), 7.48-7.23 (m, 7H), 7.02 (d, 1H), 6.71 (m, 2H), 5.06 (s, 2H), 4.44 (s, 2H), 3.75 (s, 3H), 3.58 (m, 2H), 3.03 (m, 2H).

### Example 11-1

### N-(2-aminoethyl)-N-[4-(benzyloxy)-3-methoxybenzyl]-2-(4-fluorophenyl)acetamide hydrochloride

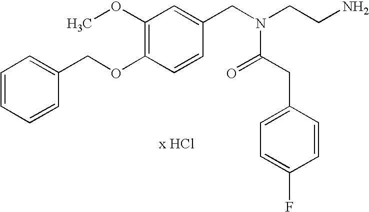

150 mg (0.287 mmol) tert-butyl(2-{[4-(benzyloxy)-3-methoxybenzyl][(4-fluorophenyl)acetyl]-amino}ethyl)carbamate are dissolved in 1 ml dioxane and treated at rt with 0.72 ml 4M solution of hydrochloric acid in dioxane. After stirring for 1 h at rt the mixture is dissolved in methanol and purified directly by RP HPLC chromatography (gradient water/acetonitrile) to afford 46.7 mg (36% of th.) of the title compound.

LC-MS (method 4-1): Rₜ=1.78 min, m/z=423 (M−HCl+H)⁺

¹H-NMR (400 MHz, DMSO-d₆): δ=(signals indicate presence of rotamers in a 1:1 ratio) 8.11/7.89 (2bs, 3H), 7.48-7.30 (m, 6H), 7.25 (t, 1H), 7.18-7.07 (m, 2H), 7.02/6.96 (2d, 1H), 6.82-6.68 (m, 2H), 5.07/5.04 (2s, 2H), 4.60/4.48 (2s, 2H), 3.81/3.78 (2s, 2H), 3.73/3.69 (2s, 3H), 3.51/3.47 (2t, 2H), 3.00/2.89 (2m, 2H).

### Example 12-1

### N-(2-aminoethyl)-N-[4-(benzyloxy)-3-chlorobenzyl]-4-fluorobenzamide

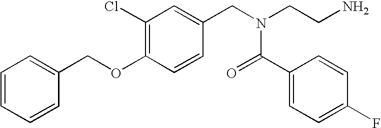

1.1 g (0.8 mmol) N-[4-(benzyloxy)-3-chlorobenzyl]-1,2-diaminoethane trityl-resin are dissolved in 8 ml dichloromethane, 0.66 ml (4 mmol) N,N-diisopropylethylamine and 0.208 ml (1.76 mmol) 4-fluorobenzoyl chloride are added. The mixture is agitated for 1 h at rt, before diluted with dichloromethane and filtrated. The resin is washed with dichloromethane, DMF/dichloromethane (1:1 mixture) and 4 times with dichloromethane. The resin is suspended in 4 ml dichloromethane and treated at rt with 0.35 ml TFA while gentile agitated. After 30 min the mixture is diluted with dichloromethane and filtered. For a second cleavage cycle the resin is again suspended in 4 ml dichloromethane and treated at rt with 0.35 ml TFA while gentile agitated. After 30 min the mixture is diluted with dichloromethane and filtered. All filtrates are combined and washed with sat. sodium bicarbonate solution and water, dried over magnesium sulfate and concentrated in vacuo. The crude product is purified by chromatography on silica gel (gradient: dichloromethane to dichloromethane/methanol 100:1-50:1-20:1) to yield 163 mg of the title compound.

LC-MS (method 4-1): Rₜ=1.87 min, m/z=413 (M+H)⁺

¹H-NMR (300 MHz, DMSO-d₆): 6=(signals indicate presence of rotamere in a 5:1 ratio) 7.55-7.15 (m, 12H), 5.21/5.18 (2s, 2H), 4.70-4.33 (m, 2H), 3.30-3.02 (m, 2H), 2.80-2.55 (m, 2H), 1.38 (m, 2H).

### Example 13-1

### N-(2-aminoethyl)-N-[4-(benzyloxy)-3-chlorobenzyl]-2-(4-fluorophenyl)acetamide

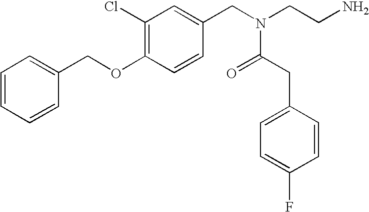

271.3 mg (1.76 mmol) 4-fluorophenyl acetic acid, 281 mg (2.08 mmol) HOBT and 0.344 ml (2.08 mmol) N,N-diisopropylethylamine are dissolved in 2 ml DMF, cooled to 0° C. and treated with 0.3 ml (1.92 mmol) diisopropylcarbodiimide. The cooling bath is removed and after 5 min the solution is transferred into a suspension of N-[4-(benzyloxy)-3-chlorobenzyl]-1,2-diaminoethane trityl-resin (0.8 mmol loading calculated) in 4 ml dichloromethane. The mixture is agitated over night at rt, before diluted with dichloromethane and filtrated. The resin is washed 4 times with dichloromethane and suspended in 4 ml dichloromethane and treated at rt with 0.35 ml TFA while gentile agitated. After 30 min the mixture is diluted with dichloromethane and filtered. For a second cleavage cycle the resin is again suspended in 4 ml dichloromethane and treated at rt with 0.4 ml TFA while gentile agitated. After 30 min the mixture is diluted with dichloromethane and filtered. All filtrates are combined and washed with sat. sodium bicarbonate solution and water, dried over magnesium sulfate and concentrated in vacuo. The crude product is purified by RP HPLC (gradient water/acetonitrile) followed by chromatography on silica gel (gradient: dichloromethane to dichloromethane/methanol 100:1) to yield 85.5 mg of the title compound.

LC-MS (method 3-1): Rₜ=1.92 min, m/z=427 (M+H)⁺

### B. EVALUATION OF PHYSIOLOGICAL ACTIVITY

The potential Cold Menthol Receptor-1 (CMR-1) antagonistic activity of the compounds of the invention may be demonstrated, for example, using the following assays:

### Measurement of the Menthol-Induced Ca²⁺ Influx in BEK293 Cell Expressing CMR-1 Receptor (Assay 1).

A cell-based calcium influx assay using HEK293 cells stably expressing human CMR-1 is used to identify CMR-1 receptor-antagonists. Menthol, a CMR-1 specific agonist, is used for stimulation of these cells, inducing an increase in intracellular calcium. This menthol-induced Ca²⁺ increase is traced by fluorescence measurement. Therefore the cells are loaded with fluo4-AM prior to stimulation. For testing inhibitors the cells are preincubated with various concentrations of the compound before menthol stimulation. The potency of potential CMR-1 inhibitors is quantified by measuring decrease of fluorescence.

TABLE A

| Example | IC₅₀ [nM] |
|---|---|
| 10 | 70 |
| 17 | 1 |
| 20 | 8 |
| 38 | 40 |
| 3-1 | 1 |
| 4-1 | 150 |

### Measurement of the Menthol-Induced Ca²⁺ Influx in Primary Cultured Rat Dorsal Root Ganglia Neurons (Assay 2)

Since CMR-1 is expressed on DRG (C-fibers), in which this receptor mediates the altered afferent information in overactive bladder; primary cultures of rat DRG are used as functional in vitro test. Stimulation of the cells is done with menthol and cold and the induced calcium influx is quantified by fluorescence in the presence or absence of CMR-1 inhibitors.

Preparation of primary cultured rat DRG neurons: DRG are prepared from Zucker rats (30 days in age) and neuronal cells are dispersed in 0.1% collagenase. After removal of Schwann cells by adhering to a culture plate, non-adherent neuronal cells are recovered and cultured on laminin- and poly-D-lysine coated 384 well plates for 2 days in the presence of 50 ng/ml rat NGF and 50 μM 5-fluorodeoxyuridine.

Measurement of Ca²⁺: Rat DRG neurons are suspended in a culture medium and seeded into 384-well plates (black walled clear-base/Nalge Nunc International). Following the culture for 48 hrs the medium is changed to 2 μM Fluo-4 AM (Molecular Probes) and 0.02% Puronic F-127 in assay buffer (Hank's balanced salt solution (HBSS), 17 mM HEPES (pH7.4), 1 mM Probenecid, 0.1% bovine serum albumin (BSA)) and the cells are incubated for 60 min at 25° C. After washing twice with assay buffer the cells are incubated with a test compound or vehicle (dimethylsulfoxide) for 20 min at 25° C. The fluorescence change indicating mobilization of cytoplasmic Ca²⁺ is measured for 60 sec after the stimulation with 50 μM menthol. The fluorescence change is calculated in the samples treated with a test compound and vehicle respectively. Inhibitory effect of the compound is calculated by a comparison of the values.

### Measurement of the Micturition Frequency in Guinea Pigs In Vivo (Assay 3)

Experiments are performed according to the principles of the national law for the protection of laboratory. Female Guinea Pigs (300-350 g) are anaesthetized with urethane (1 mg/kg i.p.). A midline abdominal incision is performed, both ureters are exposed and ligated, a catheter is implanted in the bladder pole and the abdomen is closed. For administration of the compounds the vena jugularis is exposed and canulated with a catheter. After this surgery the bladder catheter is connected via a t-shaped tube to an infusion pump (Braun Perfusor® compact) and to a pressure transducer (BioResearch Center, MLT0698, Nagoya). Saline is infused and intrabladder pressure is registered. After 1 h of equilibration period and the establishment of constant voiding cycles, menthol (0.6 mM) is added to the infused saline. At this point also vehicle (control group) or CMR-1 inhibitors are administered i.v. as bolus injection. The effect of treatment on the micturition interval (corresponding to bladder capacity) and micturition pressure is calculated and compared between vehicle-treated and compound-treated groups.

### C. OPERATIVE EXAMPLES RELATING TO PHARMACEUTICAL COMPOSITIONS

The compounds according to the invention can be converted into pharmaceutical preparations as follows:

### Tablet

### Composition:

100 mg of the compound of Example 1, 50 mg of lactose (monohydrate), 50 mg of maize starch (native), 10 mg of polyvinylpyrrolidone (PVP 25) (from BASF, Ludwigshafen, Germany) and 2 mg of magnesium stearate.

Tablet weight 212 mg, diameter 8 mm, curvature radius 12 mm.

### Preparation:

The mixture of active component, lactose and starch is granulated with a 5% solution (m/m) of the PVP in water. After drying, the granules are mixed with magnesium stearate for 5 min. This mixture is moulded using a customary tablet press (tablet format, see above). The moulding force applied is typically 15 kN.

### Orally Administrable Suspension

### Composition:

1000 mg of the compound of Example 1, 1000 mg of ethanol (96%), 400 mg of Rhodigel (xanthan gum from FMC, Pennsylvania, USA) and 99 g of water.

A single dose of 100 mg of the compound according to the invention is provided by 10 ml of oral suspension.

### Preparation:

The Rhodigel is suspended in ethanol and the active component is added to the suspension. The water is added with stirring. Stirring is continued for about 6 h until the swelling of the Rhodigel is complete.

We claim:

1. A compound of the general formula (I) 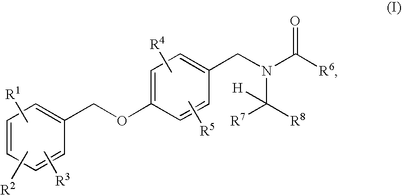
wherein
R¹ represents hydrogen or halogen,
R² represents hydrogen or halogen,
R³ represents hydrogen or halogen,
R⁴ represents chlorine, trifluoromethoxy or C₁-C₆-alkoxy,
R⁵ represents hydrogen, halogen, trifluoromethoxy, C₁-C₆-alkyl or C₁-C₆-alkoxy,
R⁶ represents C₃-C₈-alkyl, C₂-C₆-alkenyl, C₂-C₆-alkinyl, C₃-C₇-cycloalkyl, tetrahydronaphthyl, phenyl, 5- to 10-membered heteroaryl or a group of the formula —Y—R⁹,
wherein cycloalkyl can be further substituted with one to three identical or different radicals selected from the group consisting of C₁-C₄-alkyl, and
wherein phenyl and heteroaryl can be further substituted with one to three identical or different radicals selected from the group consisting of halogen, amino, hydroxy, trifluoromethyl, C₁-C₆-alkyl, C₁-C₆-alkoxy and C₁-C₆-alkylamino, and
wherein
Y represents C₁-C₄-alkandiyl,
R⁹ represents C₃-C₇-cycloalkyl, phenyl or 5- to 10-membered heteroaryl,
wherein cycloalkyl can be further substituted with one to three identical or different radicals selected from the group consisting of C₁-C₄-alkyl, and
wherein phenyl and heteroaryl can be further substituted with one to three identical or different radicals selected from the group consisting of halogen, amino, hydroxy, trifluoromethyl, C₁-C₆-alkyl, C₁-C₆-alkoxy and C₁-C₆-alkylamino,
R⁷ represents C₁-C₆-alkyl, C₃-C₇-cycloalkyl or phenyl,
wherein alkyl is further substituted with one radical selected from the group consisting of amino, mono-alkylamino, C₁-C₄-alkylcarbonylamino, C₁-C₄-alkoxycarbonylamino, phenyl or optionally C₁-C₄-alkyl substituted C₃-C₇-cycloalkyl, and
wherein cycloalkyl and phenyl are further substituted with one to three identical or different radicals selected from the group consisting of halogen, amino, hydroxy, trifluoromethyl, C₁-C₆-alkyl, C₁-C₆-alkoxy and C₁-C₆-alkylamino,
R⁸ represents hydrogen or C₁-C₄-alkyl,
or one of its salts, hydrates and/or solvates.

2. A compound of general formula (I) according to claim 1, wherein
R¹ represents hydrogen or halogen,
R² represents hydrogen or halogen,
R³ represents hydrogen,
R⁴ represents C₁-C₆-alkoxy,
R⁵ represents hydrogen,
R⁶ represents C₃-C₈-alkyl, C₂-C₆-alkenyl, C₃-C₇-cycloalkyl, tetrahydronaphthyl, phenyl, 5- to 6-membered heteroaryl or a group of the formula —Y—R⁹,
wherein cycloalkyl can be further substituted with one to three identical or different radicals selected from the group consisting of C₁-C₄-alkyl, and
wherein phenyl and heteroaryl can be further substituted with one to three identical or different radicals selected from the group consisting of halogen, trifluoromethyl, C₁-C₆-alkyl and C₁-C₆-alkoxy, and
wherein
Y represents C₁-C₄-alkandiyl,
R⁹ represents C₃-C₇-cycloalkyl, phenyl or 5- to 6-membered heteroaryl,
wherein cycloalkyl can be further substituted with one to three identical or different radicals selected from the group consisting of C₁-C₄-alkyl, and
wherein phenyl and heteroaryl can be further substituted with one to three identical or different radicals selected from the group consisting of halogen, trifluoromethyl, C₁-C₆-alkyl and C₁-C₆-alkoxy,
R⁷ represents C₁-C₄-alkyl, C₃-C₆-cycloalkyl or phenyl,
wherein alkyl is further substituted with one radical selected from the group consisting of amino, mono-alkylamino, C₁-C₄-alkoxycarbonylamino, phenyl or optionally C₁-C₄-alkyl substituted C₃-C₆-cycloalkyl, and
wherein cycloalkyl and phenyl can be further substituted with one to three identical or different radicals selected from the group consisting of amino, C₁-C₆-alkyl, C₁-C₆-alkoxy and C₁-C₆-alkylamino,
R⁸ represents hydrogen.

3. A compound of general formula (I) according to claim 1 or 2, wherein
R¹ represents hydrogen, fluorine or chlorine,
R² represents hydrogen or fluorine,
R³ represents hydrogen,
R⁴ represents methoxy,
R⁵ represents hydrogen,
R⁶ represents C₃-C₆-alkyl, C₃-C₆-cycloalkyl, tetrahydronaphthyl, phenyl, thienyl, furyl, pyrazolyl or a group of the formula —Y—R⁹,
wherein cycloalkyl can be further substituted with one or two methyl groups, and
wherein phenyl, thienyl, furyl and pyrazolyl can be further substituted with one to three identical or different radicals selected from the group consisting of fluorine, chlorine, trifluoromethyl, methyl and methoxy, and
wherein
Y represents methylen,
R⁹ represents C₃-C₆-cycloalkyl, thienyl, furyl or pyrazolyl,
wherein cycloalkyl can be further substituted with one or two methyl groups, and
wherein thienyl, furyl and pyrazolyl can be further substituted with one to three identical or different radicals selected from the group consisting of fluorine, chlorine, trifluoromethyl, methyl and methoxy,
R⁷ represents C₁-C₂-alkyl, cyclopropyl, cyclohexyl or phenyl,
wherein alkyl is further substituted with one radical selected from the group consisting of amino or tert-butoxycarbonylamino,
R⁸ represents hydrogen.

4. Process for synthesizing a compound of general formula (I) according to claim 1, by condensing a compound of general formula (II) 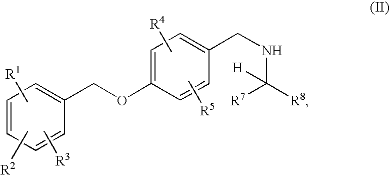
wherein R¹, R², R³, R⁴, R⁵, R⁷ and R⁸ have the meaning indicated in claim 1,
with a compound of general formula (III) 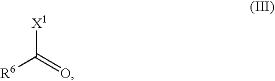
wherein R⁶ has the meaning indicated in claim 1, and
X¹ represents a leaving group, such as halogen, preferably chlorine or bromine, or hydroxy,
in the presence of a base.

5. A compound of general formula (I) according to claim 1, 2 or 3 for the treatment of diseases or disorders.

6. Use of a compound of general formula (I) according to claim 1, 2 or 3 for the preparation of medicaments.

7. Use according to claim 6 for the preparation of medicaments for the treatment of urological diseases or disorders.

8. The composition containing at least one compound of general formula (I) according to claim 1, 2 or 3 and a pharmacologically acceptable diluent.

9. A composition according to claim 8 for the treatment of urological diseases or disorders.

10. The process for the preparation of compositions according to claim 8 and 9 characterized in that the compounds of general formula (I) according to claim 1, 2 or 3 together with customary auxiliaries are brought into a suitable application form.
